(19) United States
(12) Patent Application Publication
(10) Pub. No.: US 2018/0127776 A1
(43) Pub. Date: May 10, 2018

(54) Ligand Matched Transcription Control, Control Devices, and Solute Carriers

(71) Applicant: Chimera Bioengineering, Inc., Menlo Park, CA (US)

(72) Inventors: Benjamin Wang, Menlo Park, CA (US); Gusti Zeiner, Pacifica, CA (US)

(73) Assignee: Chimera Bioengineering, Inc., Menlo Park, CA (US)

(21) Appl. No.: 15/800,621

(22) Filed: Nov. 1, 2017

Related U.S. Application Data
(60) Provisional application No. 62/417,879, filed on Nov. 4, 2016.

(51) Int. Cl. C12N 15/85; C07K 14/705; C12N 5/0789; C12N 5/0783

(52) U.S. Cl. CPC C12N 15/85; C07K 14/705; C12N 5/0647; C12N 5/0636; C12N 2510/00; C07K 14/70596; C07K 2319/03; C12N 2840/002; C12N 5/0646

(57) ABSTRACT

In an aspect, the present invention relates generally to RNA control devices, destabilizing elements (“DE”), Solute Carrier Transporters (“SLC”), and/or control regions where some or all of these control elements are ligand matched. In another aspect, the present invention relates to ligand matched control regions, SLCs and/or control devices that produce control systems with desired properties. In a further aspect, the present invention relates to use of these control systems to control expression of transgenes in eukaryotic cells including, for example, stem cells, hematopoietic cells, and host cells for gene therapy.

### FIELD OF THE INVENTION

In an aspect, the present invention relates generally to RNA control devices, destabilizing elements (“DE”), Solute Carrier Transporters (“SLC”), and/or control regions where some or all of these control elements are ligand matched. In another aspect, the present invention relates to ligand matched control regions, SLCs and/or control devices that produce control systems with desired properties. In a further aspect, the present invention relates to use of these control systems to control expression of transgenes in eukaryotic cells including, for example, stem cells, hematopoietic cells, and host cells for gene therapy.

### BACKGROUND OF THE INVENTION

Chimeric Antigen Receptors are human engineered receptors that may direct a T-cell to attack a target recognized by the CAR. For example, CAR T cell therapy has been shown to be effective at inducing complete responses against acute lymphoblastic leukemia and other B-cell-related malignancies and has been shown to be effective at achieving and sustaining remissions for refractory/relapsed acute lymphoblastic leukemia (Maude et al., NEJM, 371:1507, 2014). However, dangerous side effects related to cytokine release syndrome (CRS), tumor lysis syndrome (TLS), B-cell aplasia and on-tumor, off-target toxicities have been seen in some patients.

There are currently two extant strategies to control CAR technology. The first is an inducible “kill switch.” In this approach, one or more “suicide” genes that initiate apoptotic pathways are incorporated into the CAR construct (Budde et al. PLoS1, 2013 doi:10.1371/journal.pone.0082742). Activation of these suicide genes is initiated by the addition of AP1903 (also known as rimiducid), a lipid-permeable tachrolimus analog that initiates homodimerization of the human protein FKBP12 (Fv), to which the apoptosis-inducing proteins are translational fused. In the ideal scenario, these kill switches endeavor to sacrifice the long-term surveillance benefit of CAR technology to safeguard against toxicity. However, in vivo, these suicide switches are not likely to realize this goal, as they are operating against powerful selection pressures for CAR T-cells that do not respond to AP1903, a situation worsened by the inimical error-prone retroviral copying associated with the insertion of stable transgenes into patient T-cells. In this scenario, non-responsive CAR T-cell clones will continue to proliferate and kill target cells in an antigen-dependent manner. Thus, kill switch technology is unlikely to provide an adequate safeguard against toxicity.

The second CAR regulatory approach is transient CAR expression, which can be achieved in several ways. In one approach, T-cells are harvested from unrelated donors, the HLA genes are deleted by genome-editing technology and CAR-encoding transgenes are inserted into the genome of these cells. Upon adoptive transfer, these CAR T-cells will be recognized by the recipient's immune system as being foreign and destroyed, thus the CAR exposure in this system is transient. In another transient CAR exposure approach, mRNA of a CAR-encoding gene is introduced into harvested patient T-cells (Beatty, GL 2014. Cancer Immunology Research 2 (2): 112-20. doi:10.1158/2326-6066.CIR-13-0170). As mRNA has a short half-life and is not replicated in the cell or stably maintained, there is no permanent alteration of the CAR-expressing T-cell, thus the CAR expression and activity will be for a short period of time. However, as with the kill-switch approach, these transient CAR exposure approaches sacrifice the surveillance benefit of CARs. Additionally, with these transient systems acute toxicity can be difficult to control.

### SUMMARY OF THE INVENTION

In some embodiments, the invention relates to ligand matched control devices and solute carriers (e.g., permeases, active transporters or pores/channels) for use in controlling the expression of a desired nucleic acid. In some embodiments, the invention relates to ligand matched control of transcription (e.g., a TET ON control region), solute carriers (e.g., SLC22A6, SLC22A7, SLC22A11 for Tet), and optionally a control device that utilizes the same ligand (e.g., the Tet dependent ribozyme disclosed in Beilstein et al., ACS Synth. Biol. 4:526-534 (2016), which is incorporated by reference in its entirety for all purposes). In some embodiments, the invention relates to ligand matched control of transcription and control devices that utilizes Tet as its ligand. In some embodiments, the ligand matched control devices, control regions, and/or solute carriers are used to control the expression of a transgene that is engineered into a eukaryotic cell. In some embodiments, the ligand matched control devices, control regions, and/or solute carriers are used to control the expression of a transgene encoding a CAR. In some embodiments, ligand matched control devices, solute carriers, and transcription control are used to control the expression of a transgene (e.g., encoding a CAR). In some embodiments, the ligand matched control devices, control regions, and/or solute carriers are used to control the expression of a transgene encoding a cytokine. In some embodiments, the ligand matched control devices, control regions, and/or solute carriers are used to control the expression of a transgene encoding an effector polypeptide. In some embodiments, the ligand matched control devices, control regions, and/or solute carriers are used to control the expression of a transgene encoding a polypeptide for gene therapy. In some embodiments, the ligand matched control devices, control regions, and/or solute carriers are used to control the expression of a transgene encoding a transcriptional factor. In some embodiments, the ligand matched control devices, control regions, and/or solute carriers are used to control the expression of a transgene encoding a signal transduction polypeptide. In some embodiments, the ligand matched control devices, control regions, and/or solute carriers are used to control the expression of a transgene encoding a receptor. In some embodiments, the ligand matched control device, control regions, and/or solute carriers are used to control the expression of a transgene encoding a secreted polypeptide. In some embodiments, the ligand matched control devices, control regions, and/or solute carriers are used to control the expression of a transgene encoding an enzyme. In some embodiments, the ligand matched control devices, control regions, and/or solute carriers are used to control the expression of a transgene encoding a metabolite. In some embodiments, the ligand matched control devices, control regions, and/or solute carriers are used to control the expression of a transgene encoding a structural polypeptide.

In some embodiments, the ligand matched control devices, control regions, and/or solute carriers are used to control the expression of a CAR, DE-CAR, or Side-CAR useful in treating a malignancy including, for example, a solid tumor, a leukemia, lymphoma, myeloma, myelodysplastic syndrome, and/or myeloproliferative disease. In some embodiments, the malignancy is a multiple myeloma. In some embodiments, the malignancy is, for example, a sarcoma, carcinoma, melanoma, or blastoma. In some embodiments, the malignancy is a cancer of, for example, the adrenal glands, bile ducts, bladder, bone, brain-CNS, breast, cervix, colorectum, endometrial, esophagus, eye, gallbladder, gastrointestinal, kidney, larynx, liver, lung, nasal cavity, ovary, pancreas, pituitary prostate, salivary gland, skin, stomach, testicular, thymus, and uterine. In some embodiments, the malignancy is a CD19 and/or CD20 positive B-cell lymphoma. In some embodiments, the ligand matched control devices, control regions, and/or solute carriers are used to control the expression of a CAR, DE-CAR, or Side-CAR useful in treating an autoimmune disease, such as, for example a neurological disorder (e.g., multiple sclerosis), a rheumatological disorder (e.g., rheumatoid arthritis, systemic sclerosis, systemic lupus), a hematological immunocytopenia (pure red cell aplasia, immune thrombopenia, pure white cell aplasia), or a gastrointestinal disorder (inflammatory bowel disease).

In some embodiments, the ligand matched control devices, control regions, and/or solute carriers are used to control the expression of a transgene useful in therapies to treat, for example, amyotrophic lateral sclerosis (ALS), type I diabetes, Parkinson's disease, Alzheimer's, cardiac diseases, osteoarthritis, cancer, stroke, and wound repair. In some embodiments, the ligand matched control devices, control regions, and/or solute carriers are used to control the expression of a transgene useful in gene replacement therapies to treat, for example, hemophilia, β-thalassemia, Sanfilippo syndrome, macular degeneration, cystic fibrosis, amyotrophic lateral sclerosis (ALS), severe combined immunodeficiency (SCID), and chronic granulomatous disease.

In some embodiments, the invention relates to methods for making new RNA molecules, new RNA control devices and new destabilizing elements (DEs). In some embodiments, the invention relates to new RNA control devices and new destabilizing elements made by the methods of the invention. In some embodiments, the novel control device (RNA control device, DE, or Side CAR) shares ligand specificity with a solute carrier transporter (SLC). In some embodiments, the control device is engineered to have ligand specificity that overlaps with the ligand specificity of an SLC. In some embodiments, binding of ligand inactivates the RNA control device and/or the DE. In these embodiments, the RNA control device and DE are active in the absence of ligand. In some embodiments, binding of ligand activates the RNA control device and/or the DE. In these embodiments, the RNA control device and DE are inactive in the absence of ligand. In some embodiments, the Side-CAR is dissociated in the absence of ligand and associates when ligand is present to make an active CAR. In some embodiments, the Side-CAR is associated in the absence of ligand and dissociates when ligand is added inactivating the Side-CAR.

In some embodiments, the invention relates to the use of a SLC that is ligand matched with a RNA molecule, RNA control device, destabilizing element (DE), and/or Side-CAR in Smart-CAR, DE-CAR, Smart-DE-CAR, and/or Side-CAR constructs. In some embodiments, the invention relates to the use of new RNA molecules, RNA control devices, and/or new destabilizing elements in Smart CAR chassis, DE-CAR chassis, Smart-DE-CAR chassis, and/or Side CAR chassis. In some embodiments, the chassis are combined with an antigen binding domain to form CAR, Smart CAR, DE-CAR, Smart-DE-CAR, and/or Side CAR constructs with the new RNA molecules, RNA control devices, and/or new destabilizing elements.

In some embodiments, the eukaryotic cell comprises an expression vector with nucleic acids encoding the new RNA molecules, RNA control devices, DE, or RDE, and CARs and/or Side-CARs of the invention. In some embodiments, the expression vector includes a control region that is responsive to the same ligand that controls the RNA control device or DE, which ligand is transported by the ligand matched solute carrier. In some embodiments, the expression vector comprises nucleic acids encoding a polypeptide that acts at a control region, and the nucleic acid includes a control device that regulates the expression of the polypeptide that acts at the control region. In some embodiments, the eukaryotic cell of the invention is a mammalian cell. In some embodiments, the eukaryotic cell is a human cell or a murine cell. In some embodiments, the eukaryotic cell is a cell within the hematopoietic lineage. In some embodiments, the eukaryotic cell is a T-lymphocyte, a natural killer cell, a B-lymphocyte, or a macrophage.

In some embodiments, polynucleotides encoding the RNA molecules, RNA control devices, DE or RDE, transgene, and SLC is/are integrated into a chromosome of the eukaryotic cell. In some embodiments, the polynucleotide encoding the RNA molecules, RNA control devices or DE, transgene, and SLC is present in the eukaryotic cell extra-chromosomally. In some embodiments, the polynucleotide encoding the RNA molecules, RNA control devices or DE, transgene, and SLC is integrated using a genome editing enzyme (CRISPR, TALEN, Zinc-Finger nuclease), and appropriate nucleic acids (including nucleic acids encoding RNA molecules, RNA control devices or DE, and transgene). In an embodiment, the genome editing enzymes and nucleic acids integrate the nucleic acid encoding the RNA molecules, RNA control devices or DE, transgene, and SLC at a genomic safe harbor site, such as, for example, the CCR5, AAVS1, human ROSA26, or PSIP1 loci. In some embodiments, the eukaryotic cell is a human T-lymphocyte and the nucleic acid encoding the RNA molecules, RNA control devices or DE, transgene, and SLC is integrated at the CCR5 or PSIP1 loci.

### DETAILED DESCRIPTION OF THE INVENTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, an “actuator element” is defined to be a domain that encodes the system control function of the RNA control device. In some embodiments, the actuator domain encodes the gene-regulatory function.

As used herein, an “antibody” is defined to be a protein functionally defined as a ligand-binding protein and structurally defined as comprising an amino acid sequence that is recognized by one of skill as being derived from the variable region of an immunoglobulin. An antibody can consist of one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes include the kappa, lambda, alpha, gamma, delta, epsilon and mu constant region genes, as well as myriad immunoglobulin variable region genes and multiple D-segments and J-segments. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies exist as intact immunoglobulins, as a number of well characterized fragments produced by digestion with various peptidases, or as a variety of fragments made by recombinant DNA technology. Antibodies can derive from many different species (e.g., rabbit, sheep, camel, human, or rodent, such as mouse or rat), or can be synthetic. Antibodies can be chimeric, humanized, or humaneered. Antibodies can be monoclonal or polyclonal, multiple or single chained, fragments or intact immunoglobulins.

As used herein, an “antibody fragment” is defined to be at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either V_{L} or V_{H}), camelid VHH domains, and multi-specific antibodies formed from antibody fragments. The term “scFv” is defined to be a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

As used herein, an “antigen” is defined to be a molecule that provokes an immune response. This immune response may involve either antibody production, or the activation of specific immunologically-competent cells, or both. The skilled artisan will understand that any macromolecule, including, but not limited to, virtually all proteins or peptides, including glycosylated polypeptides, phosphorylated polypeptides, and other post-translation modified polypeptides including polypeptides modified with lipids, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or can be a macromolecule besides a polypeptide. Such a biological sample can include, but is not limited to a tissue sample, a tumor sample, a cell or a fluid with other biological components.

As used herein, the term “B-cell” or “B-lymphocyte” are used interchangeably and relate to lymphocytes that produce antibodies. As used herein, B-cells include pro B-cells, pre B-cells, immature B-cells, activated B-cells, plasma cells, memory B-cells and other cells within the B-cell lineage.

As used herein, the terms “Chimeric Antigen Receptor” and the term “CAR” are used interchangeably. As used herein, a “CAR” is defined to be a fusion protein comprising antigen recognition moieties and cell-activation elements.

As used herein, a “CAR T-cell” or “CAR T-lymphocyte” are used interchangeably, and are defined to be a T-cell containing the capability of producing CAR polypeptide, regardless of actual expression level. For example a cell that is capable of expressing a CAR is a T-cell containing nucleic acid sequences for the expression of the CAR in the cell.

As used herein, a “control device” is defined to be an RNA control device, a destabilizing element, a Side CAR, and/or an RNA destabilizing element.

As used herein, a “control region” is defined as a nucleic acid that binds regulatory elements involved with transcription. Typical regulatory elements include activators, repressors, RNA polymerase, promoters, and other transcription factors.

As used herein, a “destabilizing element” or a “DE” or a “Degron” are used interchangeably, and are defined to be a polypeptide sequence that is inducibly resistant or susceptible to degradation in the cellular context by the addition or subtraction of a ligand, and which confers this stability modulation to a co-translated polypeptide to which it is fused in cis.

As used herein, an “effective amount” or “therapeutically effective amount” are used interchangeably, and defined to be an amount of a compound, formulation, material, or composition, as described herein effective to achieve a particular biological result.

As used herein, an “epitope” is defined to be the portion of an antigen capable of eliciting an immune response, or the portion of an antigen that binds to an antibody. Epitopes can be a protein sequence or subsequence that is recognized by an antibody.

As used herein, an “expression vector” and an “expression construct” are used interchangeably, and are both defined to be a plasmid, virus, or other nucleic acid designed for protein expression in a cell. The vector or construct is used to introduce a gene into a host cell whereby the vector will interact with polymerases in the cell to express the protein encoded in the vector/construct. The expression vector and/or expression construct may exist in the cell extrachromosomally or integrated into the chromosome. When integrated into the chromosome the nucleic acids comprising the expression vector or expression construct will be an expression vector or expression construct.

As used herein, a “hematopoietic cell” is defined to be a cell that arises from a hematopoietic stem cell. This includes but is not limited to myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

As used herein, a “RNA control device” is defined to be an RNA molecule that can adopt different structures and behaviors that correspond to different gene regulatory activities.

As used herein, a “single chain antibody” (scFv) is defined as an immunoglobulin molecule with function in antigen-binding activities. An antibody in scFv (single chain fragment variable) format consists of variable regions of heavy (V_{H}) and light (V_{L}) chains, which are joined together by a flexible peptide linker.

As used herein, a “T-lymphocyte” or T-cell” is defined to be a hematopoietic cell that normally develops in the thymus. T-lymphocytes or T-cells include, but are not limited to, natural killer T cells, regulatory T cells, helper T cells, cytotoxic T cells, memory T cells, gamma delta T cells and mucosal invariant T cells.

As used herein, a “Solute Carrier,” “Solute Carrier Transporter,” and “SLC” are used interchangeably and are defined to be membrane bound proteins that transport a wide variety of substrates across membranes in eukaryotes and prokaryotes. Included within SLC are, for example, channels, pores, electrochemical potential driven transporters, primary active transporters, group translocators, electron carriers, ATP powered pumps, ion channels, and transporters, including uniporters, symporters, and antiporters.

As used herein, “transfected” or “transformed” or “transduced” are defined to be a process by which exogenous nucleic acid is transferred or introduced into a host cell. A “transfected” or “transformed” or “transduced” cell is one which has been transfected, transformed or transduced with exogenous nucleic acid. The cell includes the primary subject cell and its progeny.

### Destabilizing Elements

Destabilizing elements (DE) are stability-affecting polypeptides capable of interacting with a small-molecule ligand, the presence, absence, or amount of which ligand is used to modulate the stability of the DE-polypeptide of interest. In some embodiments, the polypeptide of interest is an immunomodulatory polypeptide. In some embodiments, the polypeptide of interest is a CAR. In some embodiments, binding of ligand by a DE-CAR reduces the degradation rate of the DE-CAR polypeptide in the eukaryotic cell. In some embodiments, binding of ligand by the DE-CAR increases the degradation rate of the DE-CAR in the eukaryotic cell.

Destabilizing elements or DEs useful in the present invention are described in U.S. patent application Ser. No. 15/070,352 filed on Mar. 15, 2016, which is incorporated by reference in its entirety for all purposes. For example, USSN 15/070,352 describes DEs derived from variants of the FKBP protein, variants of the DHFR protein, variant estrogen receptor binding domain (ERBD), and variant phototropin 1 of Avena sativa (AsLOV2). Other examples of variant FKBP nucleic acids and polypeptides are described in US published patent application 20120178168 A1 published on Jul. 12, 2012, which is hereby incorporated by reference in its entirety for all purposes. Other examples of variant DHFR nucleic acids and polypeptides are described in US published patent application 20120178168 A1 published on July 12, 2012, which is hereby incorporated by reference in its entirety for all purposes. Other examples of variant ERBD nucleic acids, polypeptides, and ligands are described in published US patent application 20140255361, which is hereby incorporated by reference in its entirety for all purposes. Other examples of variant AsLOV2 DEs are described in Bonger et al., ACS Chem. Biol. 2014, vol. 9, pp. 111-115, and Usherenko et al., BMC Systems Biology 2014, vol. 8, pp. 128-143, which are incorporated by reference in their entirety for all purposes.

Other DEs can be derived from other ligand binding polypeptides by fusing in frame a nucleic acid encoding the ligand binding polypeptide with a nucleic acid encoding a reporter. This construct is mutagenized by well-known methods, and then mutants with increased or decreased reporter activity in response to ligand binding are identified by a selection or screening. In some embodiments, variants obtained in a first round of mutagenesis and selection/screening are further mutagenized using random mutagenesis and/or creation of combinatorial libraries of the amino acid substitutions obtained in the first round of mutagenesis and/or substitution of other amino acids at the positions identified in the first round of mutagenesis. In some embodiments, the reporter polypeptide is a light emitting polypeptide such as green fluorescent polypeptide (GFP). In some embodiments, the reporter polypeptide can be used in a selection such as, for example, a reporter polypeptide that provides a cell with antibiotic resistance or the ability to grow in a certain nutrient environment or the ability to make a certain essential nutrient (e.g., the enzyme DHFR can be used in selection schemes with certain mammalian cell lines).

Other DEs can be derived from other ligand binding polypeptides using a degron as described above for ERBD. In some embodiments, a degron is fused to the C-terminus of the ligand binding polypeptide. In some embodiments, the degron is fused to the N-terminus of the ligand binding polypeptide. In some embodiments, the ligand binding polypeptide is a ligand binding domain derived from the ligand binding polypeptide, or is some other truncated form of the ligand binding polypeptide that has the ligand binding property. In some embodiments, a nucleic acid encoding the ligand binding domain fused to a degron is fused in frame with a nucleic acid encoding a reporter. This construct is mutagenized by well-known methods, and then mutants with increased or decreased reporter activity in response to ligand binding are identified by a selection or screening. In some embodiments, variants obtained in a first round of mutagenesis and selection/screening are further mutagenized using random mutagenesis and/or creation of combinatorial libraries of the amino acid substitutions obtained in the first round of mutagenesis and/or substitution of other amino acids at the positions identified in the first round of mutagenesis.

Other ligand binding polypeptides from which variants can be made for use as DEs, include for example, enzymes, antibodies or antibody fragments or antibody fragments engineered by recombinant DNA methods with the variable domain, ligand binding receptors, or other proteins. Examples of enzymes include bromodomain-containing proteins, FKBP variants, or prokaryotic DHFR variants. Examples of receptor elements useful in making DEs include: variant ERBD, or other receptors that have ligands which are nontoxic to mammals, especially humans.

In some embodiments, the ligand(s) for the DE are selected for optimization of certain attributes for therapeutic attractiveness. These attributes include specificity to the target DE, affinity to the DE, bioavailability, stability, commercial availability, cost, available related chemical, bio-orthogonality, or combinations thereof. In some embodiments, the ligands are permeable to the plasma membrane, or are transported across the plasma membrane of a eukaryotic cell. In some embodiments, the ligand is orally dosable to a subject. In some embodiments, the ligand is inert (a pro-ligand) and is converted to the active ligand by, for example, chemical means, electromagnetic radiation, or metabolism by normal flora or the subject to produce the active ligand. In some embodiments, the ligand has a serum half-life greater than 1 hour, 2 hours, 4 hours, 6 hours, 8 hours, 12 hours, 24 hours, 48 hours, 96 hours or more. In some embodiments, the ligand has a serum half-life less than 96 hours, 48 hours, 24 hours, 18 hours, 12 hours, 10 hours, 8 hours, 6 hours, 4 hours, 2 hours or 1 hour or less. In some embodiments the ligand has a serum half-life between 1 and 96 hours, between 2 and 48 hours, between 8 and 36 hours, between 10 and 28 hours, between 12 and 24 hours, between 12 and 48 hours, between 8 and 48 hours or between 16 and 18 hours. In some embodiments, the ligand can cross the blood-brain barrier. In some embodiments, the ligand is small and lipophilic. In some embodiments, the ligand cannot normally exist in human bodies or be introduced by normal diet. In some embodiments, the affinity, as measured by Kd, of the ligands to the target DE is less than 1M, 500 mM, 100 mM, 50 mM, 20 mM, 10 mM, 5 mM, 1 mM, 500 μM, 100 μM, 50 μM, 20 μM, 10 μM, 5 μM, 1 μM, 500 nM, 100 nM, 50 nM, 20 nM, 10 nM, 5 nM, 4 nM, 3 nM, 2 nM, 1 nM, 0.5 nM, 0.1 nM or less. In some embodiments, the affinity, as measured by Kd, of the ligands to the target DE is between 1M and 1 pM, between 1 mM and 1 nM, between 100 uM and 1 nM, between 10 uM and 1 nM, between 10 uM and 10 nM, between 10 uM and 100 nM, between 10 uM and 1 uM and between 50 uM and 5 uM, between 1 uM and 500 nM. In some embodiments the ligand is a protein. In some embodiments, the ligand is a small molecule. In some embodiments, the ligand is a nucleic acid.

### RNA Control Devices

In some embodiments, the Ribonucleic acid (RNA) control devices of the invention exhibit tunable regulation of gene expression, design modularity, and target specificity. The RNA control devices of the invention can act to rewire information flow through cellular networks and reprogram cellular behavior in response to changes in the cellular environment. In regulating polypeptide expression, the RNA control devices of the invention can serve as synthetic cellular sensors to monitor temporal and spatial fluctuations in the levels of diverse input molecules. RNA control devices represent powerful tools for constructing ligand-controlled gene regulatory systems tailored to modulate the expression of CAR, DE-CAR, Side-CAR and/or other polypeptides of the invention in response to specific effector molecules enabling RNA regulation of target CAR, DE-CAR, Side-CAR and/or other polypeptide constructs in various living systems.

The RNA control devices of the invention may be either trans-acting or cis-acting. By trans-acting, it is meant that the RNA control device exerts its ligand-dependent activity on a molecule, e.g. another nucleic acid, that is different from the RNA control device, e.g. not linked through a phosphodiester (or equivalent) backbone linker, and even more preferably not covalently linked to the RNA control device at all. By cis-acting, it is meant that the RNA control device exerts its ligand-dependent activity on the same contiguous nucleic acid, i.e., a nucleic acid that is covalently linked to the RNA control device, e.g., through a phosphodiester (or equivalent) backbone linker.

In some embodiments, the RNA control devices of the invention comprise a regulatory element and a sensor element. In some embodiments, the RNA control devices of the invention comprise a single element with both a regulatory and sensory function. In some embodiments, the RNA control devices of the invention comprise a regulatory function and a sensory function. In some embodiments, the RNA control devices of the invention comprise a regulatory element, a sensor element, and an information transmission element (ITE) that functionally couples the regulatory element and the sensor element. In some embodiments, the ITE of the subject invention is based on, for example, a strand-displacement mechanism, an electrostatic interaction, a conformation change, or a steric effect. In some embodiments, the sensing function of the RNA control device leads to a structural change in the RNA control device, leading to altered activity of the regulatory function. Some mechanisms whereby these structural changes can occur include steric effects, hydrophobicity driven effects (log p), electrostatically driven effects, nucleotide modification effects (such as methylation, etc.), secondary ligand interaction effects and other effects. In some embodiments, a strand-displacement mechanism uses competitive binding of two nucleic acid sequences (e.g., the competing strand and the RNA control device strand) to a general transmission region of the RNA control device (e.g., the base stem of the aptamer) to result in disruption or restoration of the regulatory element in response to ligand binding to the sensor element.

In some embodiments, the sensor element-regulated nucleic acids are designed such that it can adopt at least two distinct conformations. In one conformation, the sensor element is capable of binding to a ligand, and the regulatory element may be in one activity state (e.g., more active state or less active state). In the other conformation, the sensor element is incapable of binding to the ligand, and regulatory element may be in another activity state. The conformation change of the sensor element may be transmitted through the information transmission element to the coupled regulatory element, so that the regulatory element adopts one of the two activity states depending on whether the sensor element can or cannot bind the ligand.

In some embodiments, the aptamer-regulated nucleic acid platform is fully modular, enabling ligand response and regulatory function (e.g., transcript targeting) to be engineered by swapping elements within the subject regulated nucleic acid. This provides a platform for the construction of tailor-made sensor element regulated nucleic acids for a variety of different ligands. Ligand binding of the sensor element in sensor-regulated nucleic acids is designed separately from the targeting capability of the regulatory element by swapping only the sensor element. Likewise, the targeting capability of the regulatory element can be designed separately from the ligand binding of the sensor element by swapping the regulatory element so that a different gene or molecule is targeted without affecting the sensor element. Thus, the subject sensor element-regulated nucleic acids present a powerful, flexible method of tailoring spatial and temporal gene expression in both natural and engineered contexts.

In some embodiments, the RNA control devices are cis-acting RNA sequences that regulate the production of cognate protein encoded by a messenger RNA (mRNA). In some embodiments RNA control devices comprise RNA with sequences that enable direct or indirect binding of a ligand. In some embodiments, binding of a ligand to the RNA control device increases or decreases the amount of protein translated from the mRNA. In some embodiments, RNA control devices comprise riboswitches which are segments of mRNA that bind a small molecule.

An example of an RNA control device is the theophylline responsive switch, comprising an aptamer (a ligand binding component) and hammerhead ribozyme (gene regulating component) (Win and Smolke 2007 PNAS 104 (36): 14283-88, which is hereby incorporated by reference in its entirety for all purposes). Upon aptamer binding of theophylline, the ribozyme becomes inactive and enables the expression of the desired transgene. In the absence of theophylline the ribozyme self cleaves, leading to nuclease driven degradation of the mRNA, inhibiting translation of the mRNA into protein.

In some embodiments, the RNA control device comprises a sensor element and a regulatory element. In some embodiments the sensor element is an RNA aptamer. In some embodiments, the RNA control device comprises more than one sensor element. In some embodiments the regulatory element is a ribozyme. In some embodiments the ribozyme is a hammerhead ribozyme. In some embodiments, the ribozyme is a hairpin ribozyme, or a hepatitis delta virus (HDV) ribozyme, or a Varkud Satellite (VS) ribozyme, or a glmS ribozyme. In other embodiments the ribozyme is a ribozyme known in the art.

In some embodiments, the RNA control device is embedded within a nucleic acid that encodes a transgene. In some embodiments the transgene of interest encodes a chimeric antigen receptor, a DE-chimeric antigen receptor, or a Side CAR.

In some embodiments an RNA control device or devices are embedded within a DNA sequence. In some embodiments, the RNA control device is encoded for in messenger RNA. In some embodiments multiple RNA control devices are encoded in cis with a transgene-encoding mRNA. In some embodiments, the RNA control device is repeated. In some embodiments the nucleic acid that is used to encode the RNA control device is repeated. By including multiple RNA control devices, sensitivity and dose response may be tailored or optimized. In some embodiments multiple RNA control devices are included, with each RNA control device being specific for a different ligand. This embodiment can mitigate unintentional expression due to endogenously produced ligands that interact with the sensor element.

### RNA Control Devices: Sensor Elements

Sensor elements useful in the present invention are described in U.S. patent application Ser. No. 15/070,352 filed on Mar. 15, 2016, which is incorporated by reference in its entirety for all purposes. In some embodiments, an “aptamer” is a nucleic acid molecule, such as RNA or DNA that is capable of binding to a specific molecule with high affinity and specificity (Ellington et al., Nature 346, 818-22 (1990); and Tuerk et al., Science 249, 505-10 (1990), which are hereby incorporated by reference in their entirety for all purposes). For a review of aptamers that recognize small molecules, see Famulok, Science 9:324-9 (1999), which is hereby incorporated by reference in its entirety for all purposes.

In some embodiments, the binding affinity of the aptamer for its ligand is sufficiently strong and the structure formed by the aptamer when bound to its ligand is significant enough so as to switch an RNA control device of the invention between “on” and “off” states. In some embodiments, the association constant for the aptamer and associated ligand is preferably such that the ligand functions to bind to the aptamer and have the desired effect at the concentration of ligand obtained upon administration of the ligand to a subject. For in vivo use, for example, the association constant should be such that binding occurs well below the concentration of ligand that can be achieved in the serum or other tissue, preferably well below the concentration of ligand that can be achieved intracellularly since cellular membranes may not be sufficiently permeable to allow the intracellular ligand concentration to approach the level in the serum or extracellular environment. In some embodiments, the required ligand concentration for in vivo use is also below that which could have undesired effects on the organism.

### Ligands for RNA Control Devices

RNA control devices can be controlled via the addition of exogenous or endogenous ligands. In some embodiments, the ligands are selected for optimization of certain attributes for therapeutic attractiveness. These attributes include specificity to the target RNA control device, affinity to the RNA control device, bioavailability, stability, commercial availability, cost, available related chemical, bio-orthogonality, or combinations thereof. In some embodiments, the ligands are permeable to the plasma membrane, or are transported across the plasma membrane of a eukaryotic cell. In some embodiments, the ligand for the control device is capable of being transported by an SLC. In some embodiments, the ligand for the control device also induces transcription from a control region (e.g., by binding to an activator or a repressor), and is transported by an SLC. Ligands suitable for control regions, control devices and/or SLC transport are described below, and in, for example, Kis et al., J. R. Soc. Interface 12:20141000 (2015), and Auslander et al, Treand in Biotechnol. 31:155-168 (2012), both of which are incorporated by reference in their entirety for all purposes.

In some embodiments, the ligand is orally dosable to a subject. In some embodiments, the ligand is inert (a pro-ligand) and is metabolized by normal flora or the subject to produce the active ligand. In some embodiments, the ligand has a serum half-life greater than 1 hour, 2 hours, 4 hours, 6 hours, 8 hours, 12 hours, 24 hours, 48 hours, 96 hours or more. In some embodiments, the ligand has a serum half-life less than 96 hours, 48 hours, 24 hours, 18 hours, 12 hours, 10 hours, 8 hours, 6 hours, 4 hours, 2 hours or 1 hour or less. In some embodiments the ligand has a serum half-life between 1 and 96 hours, between 2 and 48 hours, between 8 and 36 hours, between 10 and 28 hours, between 12 and 24 hours, between 12 and 48 hours, between 8 and 48 hours or between 16 and 18 hours. In some embodiments, the ligand can cross the blood-brain barrier. In some embodiments, the ligand is small and lipophilic. In some embodiments, the ligand cannot normally exist in human bodies or be introduced by normal diet. In some embodiments, the affinity, as measured by Kd, of the ligands to the target RNA control device is less than 1M, 500 mM, 100 mM, 50 mM, 20 mM, 10 mM, 5 mM, 1 mM, 500 μM, 100 μM, 50 μM, 20 μM, 10 μM, 5 μM, 1 μM, 500 nM, 100 nM, 50 nM, 20 nM, 10 nM, 5 nM, 4 nM, 3 nM, 2 nM, 1 nM, 0.5 nM, 0.1 nM or less. In some embodiments, the affinity, as measured by Kd, of the ligands to the target DE is between 1M and 1 pM, between 1 mM and 1 nM, between 100 uM and 1 nM, between 10 uM and 1 nM, between 10 uM and 10 nM, between 10 uM and 100 nM, between 10 uM and 1 uM and between 50 uM and 5 uM, between 1 uM and 500 nM. In some embodiments the ligand is a protein. In some embodiments, the ligand is a small molecule. In some embodiments, the ligand is a nucleic acid.

In some embodiments, the ligand is a naturally occurring, secreted metabolite. For example, a ligand that is uniquely produced by a tumor, or present in the tumor microenvironment is the ligand for the sensor element and binding of this ligand to the sensor element changes the activity of the RNA control device. Thus the control device is responsive and controlled through chemical signaling or proximity to a tumor.

In some embodiments, the ligand is selected for its pharmacodynamic or ADME behavior. For example ligands may be preferentially localized to specific portions of the human anatomy and physiology. For example certain molecules are preferentially absorbed or metabolized in the gut, the liver, the kidney etc. In some embodiments the ligand is selected to demonstrate preferential pharmacodynamic behavior in a particular organ. For example, it would be useful to have a ligand that preferentially localizes to the colon for a colorectal carcinoma so that the peak concentration of the ligand is at the required site, whereas the concentrations in the rest of the body is minimized, preventing undesired, nonspecific toxicity. In some embodiments the ligand is selected to demonstrate non preferential pharmacodynamic behavior. For example, for disseminated tumors like hematological malignancies, it would be useful to have non variant concentration of the ligand throughout the body.

### RNA Control Devices: Regulatory Elements

In some embodiments, the regulatory element comprises a ribozyme, or an antisense nucleic acid, or an RNAi sequence or precursor that gives rise to a siRNA or miRNA, or a shRNA or precursor thereof, or an RNAse III substrate, or an alternative splicing element, or a transcription terminator, or a ribosome binding site, or an IRES, or a polyA site. Regulatory elements useful in the present invention are described in U.S. patent application Ser. No. 15/070,352 filed on Mar. 15, 2016, which is incorporated by reference in its entirety for all purposes.

General approaches to constructing oligomers useful in antisense technology have been reviewed, for example, by van der Krol et al. (1988) Biotechniques 6:958-976; and Stein et al. (1988) Cancer Res 48:2659-2668, which are hereby incorporated by reference in their entirety for all purposes. Certain miRNAs that may be used in the invention are described in Brennecke et al., Genome Biology 4:228 (2003); Kim et al., Mol. Cells. 19:1-15 (2005), which are hereby incorporated by reference in their entirety for all purposes.

In some embodiments, the RNA control devices have multiple regulatory elements, and/or multiple sensor elements. In some embodiments, the multiple sensor elements recognize different ligands. In some embodiments, the multiple sensor elements have different effects on the regulatory element.

### RNA Destabilizing Elements

RNA destabilizing elements (RDE) are nucleic acids that affect the stability of an RNA molecule. In some embodiments, polypeptides bind to the RDE and destabilize the RNA leading to loss of function for the RNA. In some embodiments, the binding of polypeptide to the RDE stabilizes the RNA increasing the half-life of the RNA. In some embodiments, RDEs are used to control the expression of chimeric antigen receptors. In some embodiments, RDEs are used with RNA control devices, DEs, and/or Side CARs to regulate the expression of a CAR.

In some embodiments, the RDE is a Class I AU rich element (dispersed AUUUA in U rich context), a Class II AU rich element (overlapping (AUUUA)ₙ), a Class III AU rich element (U-rich stretch), a stem-loop destabilizing element (SLDE), a cytokine '3 UTR (e.g., TNFα, IL-6, IL-8), and a sequence of AUUUAUUUAUUUA. Khabar, WIREs RNA 2016, doi: 10.1002/wrna.1368 (2016); Palanisamy et al, J. Dent. Res. 91:651-658 (2012), both of which are incorporated by reference in their entirety for all purposes.

In some embodiments, the RDE is from the 3′ UTR of the gene encoding, for example, IL-1, IL-2, IL-3, IL-4, IL-6, IL-8, IL-10, GM-CSF, VEG F, PGE₂, COX-2, MMP (matrix metalloproteinases), bFGF, c-myc, c-fos, betal-AR, PTH, interferon-gamma, MyoD, p21, Cyclin A, Cyclin B1, Cyclin D1, PAI-2, NOS HANOS, TNF-alpha, interferon-alpha, bcl-2, interferon-beta, c-jun, GLUT1, p53, Myogenin, NF-M, or GAP-43. In some embodiments, the RDE is a Class I AU rich element and arises from the 3′ UTR of a gene encoding, for example, c-myc, c-fos, betal-AR, PTH, interferon-gamma, MyoD, p21, Cyclin A, Cyclin B1, Cyclin D1, PAI-2, or NOS HANOS. In some embodiments, the RDE is a Class II AU rich element and arises from the 3′ UTR of a gene encoding, for example, GM-CSF, TNF-alpha, interferon-alpha, COX-2, IL-2, IL-3, bcl-2, interferon-beta, or VEG F. In some embodiments, the RDE is a Class III AU rich element and arises from the 3′ UTR of a gene encoding, for example, c-jun, GLUT1, p53, hsp 70, Myogenin, NF-M, or GAP-43.

In some embodiments, the RDE is bound by polypeptides including, for example, ARE poly(U) binding/degradation factor (AUF-1), tristetraprolin (TTP), human antigen-related protein (HuR), butyrate response factor 1 (BRF-1), butyrate response factor 2 (BRF-2), T-cell restricted intracellular antigen-1 (TIA-1), TIA-1 related protein (TIAR), CUG triplet repeat, RNA binding protein 1 (CUGBP-1), CUG triplet repeat, RNA binding protein 2 (CUGBP-2), human neuron specific RNA binding protein (Hel-N1, Hel-N2), RNA binding proteins HuB and HuC, KH-type splicing regulatory protein (KSRP), 3-methylglutaconyl-CoA hydratase (AUH), glyceraldehyde 3-phosphate dehydrogenase (GAPDH), heat shock protein 70 (Hsp70), heat shock protein 10 (Hsp10), heterogeneous nuclear ribonucleoprotein A1 (hnRNP A1), heterogeneous nuclear ribonucleoprotein A2 (hnRNP A2), heterogeneous nuclear ribonucleoprotein A3 (hnRNP A3), heterogeneous nuclear ribonucleoprotein C (hnRNP C), heterogeneous nuclear ribonucleoprotein L (hnRNP L), Bcl-2 AU-rich element RNA binding protein (TINO), and Poly(A) Binding Protein Interacting Protein 2 (PAIP2).

In some embodiments, one RNA binding protein binds to the RDE and this protein binding increases the rate of RNA degradation. In some embodiments, one RNA binding protein binds to the RDE and this protein binding decreases the rate of degradation of the RNA. In some embodiments, more than one RNA binding protein binds to the RDE. In some embodiments, more than one RNA binding protein binds to more than one RDE In some embodiments, binding of one or more of the RNA binding proteins increases the degradation rate of the RNA. In some embodiments, binding of one or more of the RNA binding proteins decreases the degradation rate of the RNA. In some embodiments, some of the RNA binding proteins that bind the RDE increase degradation and some of the RNA binding proteins that bind the RDE decrease degradation, so that the stability of the RNA is dependent of the relative binding of the two RNA binding proteins. In some embodiments, proteins bind to the RDE binding proteins and modulate the stability effect of the RNA binding protein. In some embodiments, binding of a protein to the RNA binding protein increases RNA stability. In some embodiments, binding of a protein to the RNA binding protein decreases RNA stability. In some embodiments, the RNA has multiple RDEs and is bound by the proteins HuR and TTP. In some embodiments, the HuR protein stabilizes the RNA and the TTP protein destabilizes the RNA. In some embodiments, the RNA has at least one RDE and interacts with the proteins KSRP, TTP and HuR. In some embodiments, KSRP destabilizes the RNA and competes for binding with the HuR protein that stabilizes the RNA. In some embodiments, the KSRP protein binds to the RDE and destabilizes the RNA and the TTP protein binds to KSRP and prevents degradation of the RNA.

In some embodiments, the RDE is a Class II AU rich element, and the RNA binding protein is GAPDH. In some embodiments, the Class II AU rich element is AUUUAUUUAUUUA. In some embodiments, the Class II AU rich element and GADPH are used to effect the expression of a CAR, Smart CAR, DE-CAR, Smart-DE-CAR, and/or Side-CAR. In some embodiments, the Class II AU rich element and GADPH are used to effect the expression of a CAR, Smart CAR, DE-CAR, Smart-DE-CAR, and/or Side-CAR in a T-lymphocyte. In some embodiments, the Class II AU rich element and GADPH are used to effect the expression of a CAR, Smart CAR, DE-CAR, Smart-DE-CAR, and/or Side-CAR in a CD8+ T-lymphocyte. In some embodiments, the Class II AU rich element and GADPH are used to effect the expression of a CAR, Smart CAR, DE-CAR, Smart-DE-CAR, and/or Side-CAR in a CD4+ T-lymphocyte. In some embodiments, the Class II AU rich element and GADPH are used to effect the expression of a CAR, Smart CAR, DE-CAR, Smart-DE-CAR, and/or Side-CAR in a natural killer cell.

### Chimeric Antigen Receptors

In some embodiments, chimeric antigen receptors (CARs) are fused proteins comprising an extracellular antigen-binding/recognition element, a transmembrane element that anchors the receptor to the cell membrane and at least one intracellular element. These CAR elements are known in the art, for example as described in patent application US20140242701, which is incorporated by reference in its entirety for all purposes herein. In some embodiments, the CAR of the invention is a recombinant polypeptide construct comprising at least an extracellular antigen binding element, a transmembrane element and an intracellular signaling element comprising a functional signaling element derived from a stimulatory molecule. In some embodiments, the stimulatory molecule is the zeta chain associated with the T cell receptor complex. In some embodiments, the cytoplasmic signaling element further comprises one or more functional signaling elements derived from at least one costimulatory molecule. In some embodiments, the costimulatory molecule is chosen from 4-1BB (i.e., CD137), CD27 and/or CD28. In some embodiments, the CAR comprises a chimeric fusion protein comprising an extracellular antigen recognition element, a transmembrane element and an intracellular signaling element comprising a functional signaling element derived from a stimulatory molecule. In some embodiments, the CAR comprises a chimeric fusion protein comprising an extracellular antigen recognition element, a transmembrane element and an intracellular signaling element comprising a functional signaling element derived from a co-stimulatory molecule and a functional signaling element derived from a stimulatory molecule. In some embodiments, the CAR comprises a chimeric fusion protein comprising an extracellular antigen recognition element, a transmembrane element and an intracellular signaling element comprising two functional signaling elements derived from one or more co-stimulatory molecule(s) and a functional signaling element derived from a stimulatory molecule. In some embodiments, the CAR comprises a chimeric fusion protein comprising an extracellular antigen recognition element, a transmembrane element and an intracellular signaling element comprising at least two functional signaling elements derived from one or more co-stimulatory molecule(s) and a functional signaling element derived from a stimulatory molecule. In some embodiments, the CAR comprises an optional leader sequence at the amino-terminus (N-term) of the CAR fusion protein. In some embodiments, the CAR further comprises a leader sequence at the N-terminus of the extracellular antigen recognition element, wherein the leader sequence is optionally cleaved from the antigen recognition element (e.g., a scFv) during cellular processing and localization of the CAR to the cellular membrane.

### Chimeric Antigen Receptor—Extracellular Element

Extracellular elements useful in the present invention are described in U.S. patent application Ser. No. 15/070,352 filed on Mar. 15, 2016, which is incorporated by reference in its entirety for all purposes.

In some embodiments, the extracellular element(s) can be obtained from the repertoire of antibodies obtained from the immune cells of a subject that has become immune to a disease, such as for example, an infectious disease, cancer, or other diseases. In some embodiments, a library of extracellular element-CARs is made from the repertoire of antibodies obtained from the immune cells of a subject that has become immune to a disease. In some embodiments, the subject has become immune to an infectious disease. In some embodiments, the extracellular element may consist of an Ig heavy chain which may in turn be covalently associated with Ig light chain by virtue of the presence of CH1 and hinge regions, or may become covalently associated with other Ig heavy/light chain complexes by virtue of the presence of hinge, CH2 and CH3 domains. In some embodiments, the extracellular element(s) can be obtained from the repertoire of T-cell receptors obtained from the immune cells of a subject that has become immune to a disease. In some embodiments, a library of extracellular element-CARs is made from the repertoire of T-cell receptors obtained from the immune cells of a subject that has become immune to a disease.

As described in U.S. Pat. Nos. 5,359,046, 5,686,281 and 6,103,521 (which are hereby incorporated by reference in their entirety for all purposes), the extracellular element may be obtained from any of the wide variety of extracellular elements or secreted proteins associated with ligand binding and/or signal transduction. In some embodiments, the extracellular element is part of a protein which is monomeric, homodimeric, heterodimeric, or associated with a larger number of proteins in a non-covalent complex.

In some embodiments, there is provided a Smart CAR, DE-CAR, Smart-DE-CAR, and/or Side-CAR capable of binding to an antigen derived from Retroviridae (e.g. human immunodeficiency viruses such as HIV-1 and HIV-LP), Picornaviridae (e.g. poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae [e.g. type 1 and type 2 herpes simplex virus (HSV), varicella-zoster virus, cytomegalovirus (CMV), and herpes virus], Poxviridae (e.g. smallpox virus, vaccinia virus, and pox virus), or hepatitis C virus.

In some embodiments, antigens specific for infectious diseases targeted by the Smart CAR(s), DE-CAR(s), Smart-DE-CAR(s), and/or Side-CARs of the invention include but are not limited to any one or more of anthrax toxin, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α. Other antigens specific for infectious diseases will be apparent to those of skill in the art and may be used in connection with alternate embodiments of the invention.

In some embodiments, there is provided a Smart CAR, DE-CAR, Smart-DE-CAR, and/or Side CAR capable of binding to an antigen associated with a bacterial strain of Staphylococci, Streptococcus, Escherichia coli, Pseudomonas, or Salmonella. In some embodiments, a phagocytic immune cell is engineered with a Smart CAR, DE-CAR, Smart-DE-CAR, and/or Side CAR specific for these or other pathogenic bacteria. Such Smart CAR, DE-CAR, Smart-DE-CAR, and/or Side CAR engineered immune cells are useful in treating bacterial infections. Examples of bacterial pathogens that can be targeted by such Smart CAR(s), DE-CAR(s), Smart-DE-CAR(s), and/or Side-CARs include, Staphylococcus aureus, Neisseria gonorrhoeae, Streptococcus pyogenes, Group A Streptococcus, Group B Streptococcus (Streptococcus agalactiae), Streptococcus pneumoniae, and Clostridium tetani. In some embodiments, there is provided a Smart CAR, DE-CAR, Smart-DE-CAR, and/or Side CAR capable of binding to an antigen found on host cells infected with an infectious pathogen (e.g., a virus, a bacteria, a protozoan, or a fungus). Examples of bacterial pathogens that may infect host cells include, Helicobacter pyloris, Legionella pneumophilia, a bacterial strain of Mycobacteria sps. (e.g. M tuberculosis, M avium, M intracellulare, M kansaii, or M gordonea), Neisseria meningitides, Listeria monocytogenes, R. rickettsia, Salmonella spp., Brucella spp., Shigella spp., or certain E. coli strains or other bacteria that have acquired genes with invasive factors. Examples of viral pathogens that may infect host cells include, Retroviridae (e.g. human immunodeficiency viruses such as HIV-1 and HIV-LP), Picornaviridae (e.g. poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae [e.g. type 1 and type 2 herpes simplex virus (HSV), varicella-zoster virus, cytomegalovirus (CMV), and herpes virus], Poxviridae (e.g. smallpox virus, vaccinia virus, and pox virus), or hepatitis C virus.

In some embodiments, there is provided a Smart CAR, DE-CAR, Smart-DE-CAR, and/or Side CAR capable of binding to a tumor antigen such as any one or more of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD21, CD22, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, alpha 5β1-integrin, integrin avβ3, MORAb-009, MS4A1, MUC1, mucin CanAg, N-glycolylneuraminic acid, NPC-1C, PDGF-Rα, PDL192, phosphatidylserine, prostatic carcinoma cells, RANKL, RON, ROR1, SCH 900105, SDC1, SLAMF7, TAG-72, tenascin C, TGF β2, TGF-β., TRAIL-R1, TRAIL-R2, tumor antigen CTAA16.88, VEGF-A, VEGFR-1, VEGFR2, 707-AP, ART-4, B7H4, BAGE, β-catenin/m, Bcr-abl, MN/C IX antibody, CAMEL, CAP-1, CASP-8, CD25, CDC27/m, CDK4/m, CT, Cyp-B, DAM, ErbB3, ELF2M, EMMPRIN, ETV6-AML1, G250, GAGE, GnT-V, Gp100, HAGE, HLA-A*0201-R170I, HPV-E7, HSP70-2M, HST-2, hTERT (or hTRT), iCE, IL-2R, IL-5, KIAA0205, LAGE, LDLR/FUT, MAGE, MART-1/melan-A, MART-2/Ski, MC1R, myosin/m, MUM-1, MUM-2, MUM-3, NA88-A, PAP, proteinase-3, p190 minor bcr-abl, Pml/RARα, PRAME, PSA, PSM, PSMA, RAGE, RU1 or RU2, SAGE, SART-1 or SART-3, survivin, TPI/m, TRP-1, TRP-2, TRP-2/INT2, WT1, NY-Eso-1 or NY-Eso-B or vimentin. Other antigens specific for cancer will be apparent to those of skill in the art and may be used in connection with alternate embodiments of the invention.

In some embodiments, antigens specific for senescent cells are targeted by the CAR, DE-CAR, Smart-DE-CAR, and/or Side-CARs of the invention include but are not limited to any one or more of DEP1, NTAL, EBP50, STX4, VAMP3, ARMX3, B2MG, LANCL1, VPS26A, or PLD3. Other antigens specific for senescent cells will be apparent to those of skill in the art and may be used in connection with alternate embodiments of the invention. See, e.g., Althubiti et al., Cell Death and Disease vol. 5, p. e1528 (2014), which is incorporated by reference in its entirety for all purposes.

Other targets for extracellular elements are described in U.S. patent application Ser. No. 15/070,352 filed on Mar. 15, 2016, which is incorporated by reference in its entirety for all purposes.

### Intracellular Element

In some embodiments, the intracellular element is a molecule that can transmit a signal into a cell when the extracellular element of the Smart CAR, DE-CAR, Smart-DE-CAR, and/or Side CAR binds to (interacts with) an antigen. In some embodiments, the intracellular signaling element is generally responsible for activation of at least one of the normal effector functions of the immune cell in which the Smart CAR, DE-CAR, Smart-DE-CAR, and/or Side CAR has been introduced. The term “effector function” refers to a specialized function of a cell. Effector function of a T cell, for example, may be cytolytic activity or helper activity including the secretion of cytokines. Thus the term “intracellular signaling element” refers to the portion of a protein which transduces the effector function signal and directs the cell to perform a specialized function. While the entire intracellular signaling domain can be employed, in many cases the intracellular element or intracellular signaling element need not consist of the entire domain. To the extent that a truncated portion of the intracellular signaling domain is used, such truncated portion may be used as long as it transduces the effector function signal. The term intracellular signaling element is thus also meant to include any truncated portion of the intracellular signaling domain sufficient to transduce the effector function signal. Examples of intracellular signaling elements for use in the Smart CAR, DE-CAR, Smart-DE-CAR, and/or Side CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

Intracellular elements and combinations polypeptides useful with or as intracellular elements are described in U.S. patent application Ser. No. 15/070,352 filed on Mar. 15, 2016, which is incorporated by reference in its entirety for all purposes.

### Transmembrane Element and Spacer Element

The Smart CAR, DE-CAR, Smart-DE-CAR, and/or Side CAR of the present invention comprises a transmembrane element. The transmembrane element is attached to the extracellular element of the Smart CAR, DE-CAR, Smart-DE-CAR, and/or Side CAR. In some embodiments, a transmembrane element includes one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane element is associated with one of the other elements used in the Smart CAR, DE-CAR, Smart-DE-CAR, and/or Side CAR. In some embodiments, the transmembrane element is selected or modified by amino acid substitution to avoid binding of such elements to the transmembrane elements of the same or different surface membrane proteins, e.g., to minimize interactions with other members of the receptor complex. In some embodiments, the transmembrane element is capable of homodimerization with another Smart CAR, DE-CAR, Smart-DE-CAR, and/or Side CAR on the cell surface. In some embodiments, the amino acid sequence of the transmembrane element may be modified or substituted so as to minimize interactions with the binding elements of the native binding partner present in the same cell.

The transmembrane element may be contributed by the protein contributing the multispecific extracellular inducer clustering element, the protein contributing the effector function signaling element, the protein contributing the proliferation signaling portion, or by a totally different protein. For the most part it will be convenient to have the transmembrane element naturally associated with one of the elements. In some cases it will be desirable to employ the transmembrane element of the ζ, η FcϵR1γ chains which contain a cysteine residue capable of disulfide bonding, so that the resulting chimeric protein will be able to form disulfide linked dimers with itself, or with unmodified versions of the ζ, η FcϵR1γ chains or related proteins. In some embodiments, the transmembrane element will be selected or modified by amino acid substitution to avoid binding of such elements to the transmembrane elements of the same or different surface membrane proteins to minimize interactions with other members of the receptor complex. In some embodiments it will be desirable to employ the transmembrane element of ζ, η FcϵR1-γ and -β, MB1 (Igα), B29 or CD3-γ, or ζ, or ϵ, in order to retain physical association with other members of the receptor complex.

Transmembrane elements useful in the present invention are described in U.S. patent application Ser. No. 15/070,352 filed on Mar. 15, 2016, which is incorporated by reference in its entirety for all purposes.

Chimeric Antigen Receptors coupled with Destabilizing Elements (DE-CAR)

In some embodiments of the present invention, destabilizing elements, as described above, are combined in cis with a CAR, as described above, so that the amount of the CAR polypeptide in the eukaryotic cell is under the control of the DE. This is one embodiment of the DE-CAR of the invention. DE-CARs, selection of DEs, and use of one or multiple DEs in the present invention are described in U.S. patent application Ser. No. 15/070,352 filed on Mar. 15, 2016, which is incorporated by reference in its entirety for all purposes.

### Chimeric Antigen Receptors: Side-CARs

In some embodiments, the CARs, Smart CARs, DE-CAR, and/or Smart-DE-CARs of the invention are comprised of at least two parts which associate to form a functional CAR or DE-CAR. In some embodiments, the extracellular antigen binding element is expressed as a separate part from the transmembrane element, optional spacer, and the intracellular element of a CAR. In some embodiments, the separate extracellular binding element is associated with the host cell membrane (through a means other than a transmembrane polypeptide). In some embodiments, the intracellular element is expressed as a separate part from the extracellular element, transmembrane element, and optionally the spacer. In some embodiments the extracellular element and intracellular element are expressed separately and each has a transmembrane element, and optionally a spacer. In some embodiments, each part of the CAR or DE-CAR has an association element (“Side-CAR”) for bringing the two parts together to form a functional CAR or DE-CAR.

Side CARs, selection of Side CARs, and their use with or without a tether are described in U.S. patent application Ser. No. 15/070,352 filed on Mar. 15, 2016, which is incorporated by reference in its entirety for all purposes.

### Lymphocyte Expansion Molecule and Other Regulatory Factors

The use of DEs and/or RNA control devices in the invention to control expression of lymphocyte expansion molecule (“LEM”), IL1, IL2, IL4, IL5, IL6, IL7, IL10, IL12, IL15, GM-CSF, G-CSF, TNFα, and/or IFNγ is described in U.S. patent application Ser. No. 15/070,352 filed on Mar. 15, 2016, which is incorporated by reference in its entirety for all purposes.

### Host Cells

In the present invention, various eukaryotic cells can be used as the eukaryotic cell of the invention. In some embodiments, the eukaryotic cells of the invention are animal cells. In some embodiments, the eukaryotic cells are mammalian cells, such as mouse, rat, rabbit, hamster, porcine, bovine, feline, or canine. In some embodiments, the mammalian cells are cells of primates, including but not limited to, monkeys, chimpanzees, gorillas, and humans. In some embodiments, the mammalians cells are mouse cells, as mice routinely function as a model for other mammals, most particularly for humans (see, e.g., Hanna, J. et al., Science 318:1920-23, 2007; Holtzman, D. M. et al., JClin Invest. 103(6):R15-R21, 1999; Warren, R. S. et al., JClin Invest. 95: 1789-1797, 1995; each publication is incorporated by reference in its entirety for all purposes). Animal cells include, for example, fibroblasts, epithelial cells (e.g., renal, mammary, prostate, lung), keratinocytes, hepatocytes, adipocytes, endothelial cells, and hematopoietic cells. In some embodiments, the animal cells are adult cells (e.g., terminally differentiated, dividing or non-dividing) or embryonic cells (e.g., blastocyst cells, etc.) or stem cells. In some embodiments, the eukaryotic cell is a cell line derived from an animal or other source.

In some embodiments, the eukaryotic cells are plant cells. In some embodiments the eukaryotic cells are cells of monocotyledonous or dicotyledonous plants, including, but not limited to, maize, wheat, barley, rye, oat, rice, soybean, peanut, pea, lentil and alfalfa, cotton, rapeseed, canola, pepper, sunflower, potato, tobacco, tomato, eggplant, eucalyptus, a tree, an ornamental plant, a perennial grass, or a forage crop. In other embodiments, the eukaryotic cells are algal, including but not limited to algae of the genera Chlorella, Chlamydomonas, Scenedesmus, Isochrysis, Dunaliella, Tetraselmis, Nannochloropsis, or Prototheca, In some embodiments, the eukaryotic cells are fungi cells, including, but not limited to, fungi of the genera Saccharomyces, Klyuveromyces, Candida, Pichia, Debaromyces, Hansenula, Yarrowia, Zygosaccharomyces, or Schizosaccharomyces.

In other embodiments, the eukaryotic cells are stem cells. A person of ordinary skill in the art knows a variety of stem cell types including, for example, Embryonic Stem Cells, Inducible Pluripotent Stem Cells, Hematopoietic Stem Cells, Neural Stem Cells, Epidermal Neural Crest Stem Cells, Mammary Stem Cells, Intestinal Stem Cells, Mesenchymal stem cells, Olfactory adult stem cells, and Testicular cells.

In some embodiments, the eukaryotic cell is a cell found in the circulatory system of a mammal, including humans. Exemplary circulatory system cells include, among others, red blood cells, platelets, plasma cells, T-cells, natural killer cells, B-cells, macrophages, neutrophils, or the like, and precursor cells of the same. As a group, these cells are defined to be circulating eukaryotic cells of the invention. In some embodiments, the eukaryotic cells are derived from any of these circulating eukaryotic cells. The present invention may be used with any of these circulating cells or eukaryotic cells derived from the circulating cells. In some embodiments, the eukaryotic cell is a T-cell or T-cell precursor or progenitor cell. In some embodiments, the eukaryotic cell is a helper T-cell, a cytotoxic T-cell, a memory T-cell, a regulatory T-cell, a natural killer T-cell, a mucosal associated invariant T-cell, a gamma delta T cell, or a precursor or progenitor cell to the aforementioned. In some embodiments, the eukaryotic cell is a natural killer cell, or a precursor or progenitor cell to the natural killer cell. In some embodiments, the eukaryotic cell is a B-cell, or a plasma cell, or a B-cell precursor or progenitor cell. In some embodiments, the eukaryotic cell is a neutrophil or a neutrophil precursor or progenitor cell. In some embodiments, the eukaryotic cell is a megakaryocyte or a precursor or progenitor cell to the megakaryocyte. In some embodiments, the eukaryotic cell is a macrophage or a precursor or progenitor cell to a macrophage.

In some embodiments, a source of cells is obtained from a subject. The subject may be any living organisms. In some embodiments, the cells are derived from cells obtained from a subject. Examples of subjects include humans, dogs, cats, mice, rats, and transgenic species thereof. In some embodiments, T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In an alternative aspect, the wash solution lacks calcium and may lack magnesium or may lack many if not all divalent cations. Initial activation steps in the absence of calcium can lead to magnified activation.

Enrichment of a T cell population by negative selection can be accomplished with a combination of antibodies directed to surface markers unique to the negatively selected cells. In some embodiments, cells are enriched by cell sorting and/or selection via negative magnetic immunoadherence or flow cytometry using a cocktail of monoclonal antibodies directed to cell surface markers present on the cells. For example, to enrich for CD4+ cells, a monoclonal antibody cocktail typically includes antibodies to CD14, CD20, CD11b, CD16, HLA-DR, and CD8. In some embodiments, it may be desirable to enrich for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005, each of which is incorporated by reference in its entirety for all purposes.

In some embodiments, NK cells may be expanded in the presence of a myeloid cell line that has been genetically modified to express membrane bound IL-15 and 4-1BB ligand (CD137L). A cell line modified in this way which does not have WIC class I and II molecules is highly susceptible to NK cell lysis and activates NK cells. For example, K562 myeloid cells can be transduced with a chimeric protein construct consisting of human IL-15 mature peptide fused to the signal peptide and transmembrane domain of human CD8a and GFP. Transduced cells can then be single-cell cloned by limiting dilution and a clone with the highest GFP expression and surface IL-15 selected. This clone can then be transduced with human CD137L, creating a K562-mb15-137L cell line. To preferentially expand NK cells, peripheral blood mononuclear cell cultures containing NK cells are cultured with a K562-mb15-137L cell line in the presence of 10 IU/mL of IL-2 for a period of time sufficient to activate and enrich for a population of NK cells. This period can range from 2 to 20 days, preferably about 5 days. Expanded NK cells may then be transduced with the anti-CD19-BB-ζ chimeric receptor.

Other host cells useful in the present invention are described in U.S. patent application Ser. No. 15/070,352 filed on Mar. 15, 2016, which is incorporated by reference in its entirety for all purposes.

### Gene Therapy

In some embodiments, the novel control devices of the invention are used in gene therapy. Gene therapy may take place via a number of strategies, the most appropriate of which can be selected by a person of ordinary skill in the art depending on the particular pathogenesis of a disease. In some embodiments, gene therapy is used for making eukaryotic cells that kill specific cells in a subject to be treated. The CAR embodiments of the invention are an example of such targeted cell killing. In some embodiments, non-CAR genes are placed into eukaryotic cells and then expressed so as to cause killing of target cells. In some embodiments, cell killing can take place by a direct mechanism, e.g. if the genes which are introduced encode a lethal toxin or encode a pro-drug which confers susceptibility on the cells to killing by a subsequently administered drug. In some embodiments, cell killing can be indirect, e.g. by using immunostimulatory genes as the introduced genes in order to provoke or enhance an immune response against the target cell, or by using genes which encode a protein which causes cell death by interaction with an exogenously added molecule, (e.g. a gene encoding an enzyme that activates a pro-drug such as HSV-tk which activated GCV).

In some embodiments, the gene therapy approach involves targeted inhibition of gene expression. A variety of different techniques to specifically block the expression of a gene at the DNA, RNA or protein level are well known to a person of ordinary skill in the art, and any of these may be used in conjunction with the control devices of the invention to control the time and place of expression of molecular tools to in the cells. In some embodiments, the molecule to be introduced is a DNA sequence comprising or capable of transcribing or expressing a functional product that will inhibit gene expression at some level in the target cells, e.g. by comprising, expressing or transcribing antisense molecules, ribozymes or intracellular antibodies.

In some embodiments, gene therapy involves gene augmentation therapy, when a disease state is caused by loss of function of a gene, and the diseases may be cured by introducing extra copies of the normal gene into appropriate cells of a subject. In some embodiments, the molecule to be introduced is a gene or a portion thereof capable of expressing a functional product to compensate for a deficiency in a subject. In some embodiments, a further gene therapy approach is that of targeted mutation correction, where the introduction of a nucleic acid into the appropriate cells of a subject leads to the direct correction of a disease-causing mutation in the subject's DNA.

In some embodiments, the novel control devices of the invention control the expression of genes introduced to a eukaryotic cell so that the exogenously added gene can be expressed at a desired time and/or in a desired location in the subject.

### Stem Cell Therapy

In some embodiments, the novel control devices of the invention are used in stem cells. In some embodiments, the novel control devices control the differentiation of stem cells in a subject. In some embodiments, stem cells include, for example, Embryonic Stem Cells, Inducible Pluripotent Stem Cells, Hematopoietic Stem Cells, Neural Stem Cells, Epidermal Neural Crest Stem Cells, Mammary Stem Cells, Intestinal Stem Cells, Mesenchymal stem cells, Olfactory adult stem cells, and Testicular cells. In some embodiments, cells containing the control devices of the invention are stem cells or are derived from stem cells, including ES cells, iPS cells, or adult stem cells obtained from mammalian species, including but not limited to, human, mouse, rat, and pig. In some embodiments, stem cells are introduced into a subject directly or may be first differentiated in vitro and then introduced into a subject.

In some embodiments, the control devices of the invention control the expression of a polypeptide that causes the stem cell to differentiate into a desired cell type. In some embodiments, the polypeptide is expressed when ligand for the control device is present in the cell. In this embodiment, the stem cells can be programmed to differentiate at a desired time (or in a desired location) by controlling the timing and location of where the stem cell encounters ligand for the control device. In some embodiments, the control devices of the invention control the expression of polypeptides or RNAs that produce or cause other desired phenotypes in stem cells.

In some embodiments, the control devices and/or the SLCs of the invention are used in hematopoietic stem or progenitor cells. In some embodiments, the control devices and/or the SLCs of the invention are used in differentiated progeny obtained from the hematopoietic stem or progenitor cell. In some embodiments, hematopoietic stem cells, progenitor cells, or cells differentiated therefrom are introduced into mammals. The control devices and SLCs of the invention can be used to program activities of the hematopoietic stem cells, progenitor cells, or cells differentiated therefrom.

### Solute Carriers (SLC)

In some embodiments, Solute Carrier (SLC) transporters are used in the present invention. In some embodiments, SLCs are used in conjunction with the control means of the invention. In some embodiments, the SLC transports the ligand for the control means into a eukaryotic cell. In some embodiments, the SLC transports the ligand for the control means into mammalian cell. In some embodiments, the SLC transports ligand for the control means into a hematopoietic cell (e.g., a T-lymphocyte). In some embodiments, the SLC transports ligand for the control means into a prokaryotic cell. In some embodiments, the SLC transports the ligand for the control means out of the prokaryotic cell or eukaryotic cell.

In some embodiments, polynucleotides encoding the SLC are engineered into a eukaryotic cell. In some embodiments, polynucleotides encoding the SLC are engineered into the prokaryotic cell. In some embodiments, polynucleotides encoding the SLC are engineered into hematopoietic cells (e.g., T-lymphocytes). In some embodiments, SLCs are paired with control devices for use in a eukaryotic cell. In some embodiments, SLCs are paired with control devices for use in a prokaryotic cell. In some embodiments, SLCs are paired with control devices for use in a positive selection host cell and/or a negative selection host cell. In some embodiments, the paired SLC and control device transport and respond to the same ligand so that the SLC transports the ligand for the control device into or out of the eukaryotic or prokaryotic cell. In some embodiments, the paired SLC and control device transport and respond to the same ligand so that the SLC transports the ligand for the control device into the eukaryotic or prokaryotic cell. In some embodiments, the paired SLC and control device transport and respond to the same ligand so that the SLC transports the ligand for the control device out of the eukaryotic or prokaryotic cell.

In some embodiments, control devices are engineered to interact with and/or respond to a ligand that can be transported by an SLC. In this embodiment, the designed control device and SLC are paired together for use in a eukaryotic cell. The engineering of control devices to respond to desired ligands is described below.

In some embodiments, SLCs are eukaryotic or prokaryotic membrane proteins that transport solutes (ions, metabolites, peptides, drugs, ligands, other organic small molecules, etc.) across membranes. In some embodiments, the SLCs are active transporters and utilize energy (e.g., ATP or an ion gradient) to transport a solute (e.g., ligand) into the eukaryotic or prokaryotic cell. In some embodiments, the SLCs are passive transporters that do not utilize energy for transport of the solute (e.g., ligand). In some embodiments, exemplary SLCs are described in, for example, Lin et al., Nat. Rev. Drug Discov. 14:543-560 (2015); Hediger et al., Mol. Aspects Med. 34:95-107 (2013); Ye et al., PLoS ONE 9:e88883 (2014); Schlessinger et al., Curr. Top. Med. Chem. 13:843-856 (2013); Saier, Microbiol. 146:1775-1795 (2000); SLC Tables at slc.bioparadigms.org; HUGO Gene Nomenclature for SLCs at genenames.org/cgi-bin/genefamilies/set/752, all of which are incorporated by reference in their entirety for all purposes. In some embodiments, included within SLCs are, for example, channels, pores, electrochemical potential driven transporters, primary active transporters, group translocators, electron carriers, ATP powered pumps, ion channels, and transporters, including uniporters, symporters, and antiporters.

In some embodiments, exemplary pair of control device ligand and SLCs includes, for example, xanthine and the prokaryotic SLCs XanQ, XanP, YjcD, YgfO, YgfQ, and/or YbbY. In some embodiments, another exemplary pair of control device ligand and SLCs includes, for example, guanine, and prokaryotic SLCs YgfQ, YjcD, YbbY. Other exemplary ligands include, for example, vitamins (e.g., vitamin C), biotin, tetracycline, doxycycline, or essential amino acids (e.g., for a human, histidine, isoleucine, lysine, methionine, phenylalanine, threonine, tryptophan, and valine). Exemplary SLCs for these ligands are well-known in the art and include, for example, ascorbic acid transporters SVCT (e.g., SVCT1 and SVCT2), dehydroascorbic acid transporters (e.g., GLUT1, GLUT3 and GLUT4), biotin transporters (e.g., MCT1), tetracycline transporters (e.g., hOAT1, hOAT2, hOAT3, hOAT4, SLC22A6, SLC22A7, SLC22A11, and the like), doxycycline transporters, histidine transporters (e.g., hPHT1, SLC15A4), the L-type amino acid transporter LAT1 (transports isoleucine, leucine, valine, phenylalanine, tryptophan, methionine, and histidine) and LAT2, the arginine/lysine transporter Cat-1, neutral amino acid transporter A (SLC1A4), human alanine/serine/cysteine/threonine transporter (ASCT1), and the proline-tryptophan transporter SLC36A4. Still other exemplary ligands are found in Kis et al., J. R. Soc. Interface 12:20141000 (2015), and Auslander et al, Treand in Biotechnol. 31:155-168 (2012), both of which are incorporated by reference in their entirety for all purposes.

In some embodiments, the ligand is a hybrid or fusion ligand where one portion of the ligand interacts with the control device and another portion (or domain) of the ligand interacts with the SLC to transport the hybrid or fusion ligand. Examples of such potential hybrid or fusion ligands include, for example. biotin and avidin (or streptavidin).

### Nucleic Acids

In some embodiments, the present invention relates to the nucleic acids that encode, at least in part, the individual peptides, polypeptides, proteins, and RNA control devices of the present invention. In some embodiments, the nucleic acids may be natural, synthetic or a combination thereof. The nucleic acids of the invention may be RNA, mRNA, DNA or cDNA.

In some embodiments, the nucleic acids of the invention also include expression vectors, such as plasmids, or viral vectors, or linear vectors, or vectors that integrate into chromosomal DNA. Expression vectors can contain a nucleic acid sequence that enables the vector to replicate in one or more selected host cells. Such sequences are well known for a variety of cells. The origin of replication from the plasmid pBR322 is suitable for most Gram-negative bacteria. In eukaryotic host cells, e.g., mammalian cells, the expression vector can be integrated into the host cell chromosome and then replicate with the host chromosome. Similarly, vectors can be integrated into the chromosome of prokaryotic cells.

Expression vectors also generally contain a selection gene, also termed a selectable marker. Selectable markers are well-known in the art for prokaryotic and eukaryotic cells, including host cells of the invention. Generally, the selection gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. In some embodiments, an exemplary selection scheme utilizes a drug to arrest growth of a host cell. Those cells that are successfully transformed with a heterologous gene produce a protein conferring drug resistance and thus survive the selection regimen. Other selectable markers for use in bacterial or eukaryotic (including mammalian) systems are well-known in the art.

The expression vector for producing a heterologous polypeptide may also contain an inducible promoter that is recognized by the host RNA polymerase and is operably linked to the nucleic acid encoding the target protein. Inducible or constitutive promoters (or control regions) with suitable enhancers, introns, and other regulatory sequences are well-known in the art. In some embodiments, the inducible promoter has low basal (or constitutive) expression. In some embodiments, the inducible promoter has a large dynamic range. In some embodiments, the inducible promoter responds to a ligand by increasing expression. In some embodiments, the inducible promoter responds to a ligand by decreasing expression. In some embodiments, the inducible promoter includes, for example, the TET ON promoter, the promoters described in Kis et al., J. R. Soc. Interface 12:20141000 (2015), and Auslander et al, Treand in Biotechnol. 31:155-168 (2012), both of which are incorporated by reference in their entirety for all purposes, and other ligand inducible promoters.

In some embodiments, it may be desirable to modify the polypeptides of the present invention. One of skill will recognize many ways of generating alterations in a given nucleic acid construct to generate variant polypeptides Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques (see, e.g., Gillam and Smith, Gene 8:81-97, 1979; Roberts et al., Nature 328: 731-734, 1987, which is incorporated by reference in its entirety for all purposes). In some embodiments, the recombinant nucleic acids encoding the polypeptides of the invention are modified to provide preferred codons which enhance translation of the nucleic acid in a selected organism.

The polynucleotides of the invention also include polynucleotides including nucleotide sequences that are substantially equivalent to the polynucleotides of the invention. Polynucleotides according to the invention can have at least about 80%, more typically at least about 90%, and even more typically at least about 95%, sequence identity to a polynucleotide of the invention. The invention also provides the complement of the polynucleotides including a nucleotide sequence that has at least about 80%, more typically at least about 90%, and even more typically at least about 95%, sequence identity to a polynucleotide encoding a polypeptide recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions which can routinely isolate polynucleotides of the desired sequence identities.

Nucleic acids which encode protein analogs or variants in accordance with this invention (i.e., wherein one or more amino acids are designed to differ from the wild type polypeptide) may be produced using site directed mutagenesis or PCR amplification in which the primer(s) have the desired point mutations. For a detailed description of suitable mutagenesis techniques, see Sambrook et al., Molecular Cloning: A Laboratory Manual, Cold Spring Harbor Laboratory Press, Cold Spring Harbor, N.Y. (1989) and/or Current Protocols in Molecular Biology, Ausubel et al., eds, Green Publishers Inc. and Wiley and Sons, N.Y. (1994), each of which is incorporated by reference in its entirety for all purposes. Chemical synthesis using methods well known in the art, such as that described by Engels et al., Angew Chem Intl Ed. 28:716-34, 1989 (which is incorporated by reference in its entirety for all purposes), may also be used to prepare such nucleic acids.

In some embodiments, amino acid “substitutions” for creating variants are preferably the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

The nucleic acid of the present invention can be linked to another nucleic acid so as to be expressed under control of a suitable promoter. The nucleic acid of the present invention can be also linked to, in order to attain efficient transcription of the nucleic acid, other regulatory elements that cooperate with a promoter or a transcription initiation site, for example, a nucleic acid comprising an enhancer sequence, a polyA site, or a terminator sequence. In addition to the nucleic acid of the present invention, a gene that can be a marker for confirming expression of the nucleic acid (e.g. a drug resistance gene, a gene encoding a reporter enzyme, or a gene encoding a fluorescent protein) may be incorporated.

When the nucleic acid of the present invention is introduced into a cell ex vivo, the nucleic acid of the present invention may be combined with a substance that promotes transference of a nucleic acid into a cell, for example, a reagent for introducing a nucleic acid such as a liposome or a cationic lipid, in addition to the aforementioned excipients. Alternatively, a vector carrying the nucleic acid of the present invention is also useful. Particularly, a composition in a form suitable for administration to a living body which contains the nucleic acid of the present invention carried by a suitable vector is suitable for in vivo gene therapy.

### Cheetah: Method for Obtaining Novel Control Devices

In some embodiments, methods of the invention are used to make novel control devices. In some embodiments, the novel control device is a RNA control device. In some embodiments, the novel control device is a destabilizing element (DE). In some embodiments, a nucleic acid encoding a starting control device is placed into a selection construct. In some embodiments, the selection construct is a plasmid (that can exist episomally or integrated into a chromosome of a host cell), virus, or other construct that can propagate in an appropriate host cell. In some embodiments, the selection construct also encodes a polypeptide and expression of the polypeptide or activity of the polypeptide is under the control of the control device. In some embodiments, the polypeptide has an activity that can be used in a selection, either directly or indirectly. In some embodiments, the selection construct is subjected to conditions under which active polypeptide is needed for the selection construct to propagate (positive selection). In some embodiments, the selection construct is subjected to conditions under which the activity of the polypeptide must be suppressed in order for the selection construct to propagate (negative selection).

In some embodiments, the polypeptide encoded in the selection construct is a polymerase, a sigma factor, a repressor, a transcriptional activator, bacterial enhancer binding proteins, or other transcriptional factor. In some embodiments, the polypeptide is a polymerase including, for example, T7 RNA polymerase, T3 RNA polymerase, SP6 RNA polymerase, or other RNA polymerase with control region specificity that is different from the host cell RNA polymerase(s) specificity. In some embodiments, the polypeptide is a sigma factor including, for example, sigma 32 (RpoH the heat shock sigma factor), sigma 24 (RpoE the extreme heat sigma factor), sigma 38 (RpoS the stationary phase sigma factor), sigma 54 (RpoN the nitrogen sigma factor), sigma 19 (Fecl the ferric citrate sigma factor), or sigma 28 (RpoF the flagellar sigma factor). In some embodiments, the polypeptide is a repressor including, for example, lac repressor, methionine repressor, trp repressor, lambda C1 repressor, P22 C2 repressor, or WT1. In some embodiments, the polypeptide is a transcriptional activator including, for example, catabolite activator protein (aka CREB, cAMP response element binding protein or CRP, cAMP receptor protein), phage 169 CII protein, LdtR, Ga14, Gcn4, Gli2, or ZNF143.

In some embodiments, the selection construct can be packaged by viruses using helper virus (plasmid) based systems. In some embodiments, the construct-helper virus (helper plasmid) systems that can be used in the invention are based on bacteriophages. In some embodiments, the construct-helper virus (helper plasmid) systems that can be used in the invention are based on M13, Fd, F1, lambda, or P22. In some embodiments, the construct-helper virus (plasmid) systems that can be used in the invention are based on eukaryotic viruses. In some embodiments, the construct-helper virus (plasmid) systems that can be used in the invention are based on adenovirus, adeno associated virus, lentivirus, alphavirus, herpesvirus, or vaccinia virus. In some embodiments, construct packaging cell lines are used where the helper virus genes needed for the vector are located in the packaging cell line either episomally or chromosomally.

In some embodiments, the selection construct includes F or R factor genes needed for conjugation transfer of the selection construct to new host cells. In some embodiments, the F or R factor genes include pili, tra and replication genes. In some embodiments, some of the F or R factor genes may be carried on helper plasmids or integrated into the host chromosome (as long as these genes do not lead to surface exclusion as caused by, for example, traS and traT).

In some embodiments, the selection construct and helper constructs are used with an appropriate host cell, including for example, Escherichia coli, Salmonella, or other bacteria that can host the construct—helper system or conjugation system. In some embodiments, appropriate eukaryotic cell hosts are used for eukaryotic virus based systems, including, for example, yeast cells, HEK293 cells, 293T-cells, VERO cells, BHK-21 cells, HeLa cells, or other cells and cell lines that can be infected by the viral particles used in the construct-helper system.

In some embodiments, the polypeptide encoded by the selection construct must be expressed and active in order for the selection vector to propagate in a host cell. In some embodiments, the polypeptide encoded by the selection construct is a RNA polymerase, a sigma factor, a repressor, a transcriptional activator, bacterial enhancer binding proteins, or other transcriptional factor. In some embodiments, the RNA polymerase is a T7 RNA polymerase. In some embodiments, the sigma factor is sigma 32 (RpoH the heat shock sigma factor). In some embodiments, the RNA polymerase, sigma factor, or other transcriptional activator causes expression from a control region in a positive selection construct so that a polypeptide needed for propagation of the selection construct is expressed. In some embodiments, the polypeptide needed for propagation is a replication factor or transfer factor for the selection construct. In some embodiments, the polypeptide needed for propagation is a viral coat protein needed to make virus particles for packaging the selection construct.

In some embodiments, the polypeptide encoded by the selection construct is a repressor protein, e.g., Lac repressor. In some embodiments, repressor protein controls the expression of a polypeptide in the host cell that is needed for propagation of the selection construct. In this embodiment, expression of active repressor protein must be inhibited in order to have expression of the polypeptide needed for propagation of the selection construct. In some embodiments, the RNA control device or DE is active by adding or excluding ligand so that expression of active Lac repressor is inhibited by either degradation of the mRNA for Lac repressor by the RNA control device or degradation of the Lac repressor polypeptide by the DE. In some embodiments, this produces expression of the polypeptide needed for the selection construct to propagate, and the selection construct propagates.

In some embodiments, the selection construct (SC) enters a host cell that has a helper construct, RNA polymerase expresses a transcript from the SC encoding the control device and SC polypeptide, ligand for the control device is present in the host cell, the control device is inactive and active SC polypeptide is expressed, the SC polypeptide expresses coat protein from the positive selection construct, and the coat protein combines with other viral proteins made by the helper construct to make a viral capsid that packages replicated SC. In this embodiment, the SC selects for those control devices that bind ligand well and/or produce a robust off signal for the associated control device. In some embodiments, the concentration of ligand can be reduced selecting for control devices with increased affinity for ligand and/or improved off signal communication and/or improved off activity by the control device.

In some embodiments, the selection construct (SC) enters a host cell with a helper construct, RNA polymerase expresses a transcript from the SC encoding the control device and SC polypeptide, ligand for the control device is present in the host cell, the control device is inactive and active SC polypeptide is expressed, the SC polypeptide expresses a replication or transfer protein from the positive selection construct, and the replication or transfer protein assists in the conjugal transfer of the SC to a new host. In this embodiment, the SC selects for those control devices that bind ligand well and/or produce a robust off signal for the associated control device. In some embodiments, the concentration of ligand can be reduced selecting for control devices with increased affinity for ligand and/or improved off signal communication and/or off activity by the control device.

In some embodiments, the polypeptide encoded by the selection construct must be suppressed and inactive in order for the selection vector to propagate in a host cell. In some embodiments, the RNA polymerase, sigma factor, or other transcriptional activator causes expression from a negative selection construct so that a polypeptide which inhibits propagation of the selection construct is expressed. In some embodiments, the polypeptide encoded by the negative selection construct is a mutant replication factor that prevents replication or transfer of the selection construct (is a dominant mutant to the wild type replication factor). In some embodiments, the negative selection construct encodes a mutant viral coat protein that prevents propagation of the SC.

In some embodiments, the polypeptide encoded by the selection construct is a repressor protein, e.g., Lac repressor. In some embodiments, repressor protein controls expression of a polypeptide from the negative selection construct that prevents propagation of the selection construct. In this embodiment, expression of active repressor protein prevents expression of the propagation inhibitory polypeptide from the negative selection construct. In some embodiments, the RNA control device or DE is activated by adding or excluding ligand so that active Lac repressor is made because the RNA control device and/or DE are inhibited from acting. In some embodiments, this represses expression of the polypeptide that prevents propagation of the selection construct, and the selection construct propagates.

In some embodiments, the selection construct (SC) enters a host cell comprising a helper construct, a drift construct which expresses a coat protein, and a negative selection construct that expresses a mutant coat protein; RNA polymerase in the host cell expresses a transcript from the SC encoding the control device and SC polypeptide; the control device is active and active SC polypeptide is not expressed, the mutant coat protein is not expressed from the negative selection construct, and functional phage particles are made that package replicated SC. In this embodiment, the SC selects for those control devices that have a robust activity for preventing translation of the mRNA (RNA control device) or a robust activity for degrading the SC polypeptide (DE). In some embodiments, the ligand can be added selecting for control devices with increased activity.

In some embodiments, the selection construct (SC) enters a host cell with a helper construct, a negative selection construct, and a drift construct; RNA polymerase expresses a transcript from the SC encoding the control device and SC polypeptide; the control device is active and active SC polypeptide is not expressed, the negative selection construct is not expressed, the drift and helper constructs express the polypeptides needed for replication and/or transfer, and the SC is transferred by conjugation to a new host. In this embodiment, the SC selects for those control devices that have a robust activity for preventing translation of the mRNA (RNA control device) or a robust activity for degrading the SC polypeptide (DE). In some embodiments, the ligand for the control device can be added selecting for control devices with increased activity.

In some embodiments, a desired level of sequence change (or mutagenesis) is introduced into the control device of the selection construct at each round of selection. In some embodiments, a desired level of sequence change occurs because the selection construct goes through a single stranded phase during replication. In some embodiments, a construct encoding some or all of dnaQ926 (proofreading exonuclease mutant), umuD′ and umuC (DNA pol V), recA730, dam (methylation), seqA (replication initiation), ugi (uracil-DNA glycosylase inhibitor), cdal (cytidine deaminase), and emrR (repressor of effux pump) is included in the host cell to increase the mutagenesis rate.

In some embodiments, the positive and negative selection cycles occur in a chemostat with alternating input of host cells with positive selection constructs, and host cells with negative selection constructs. In this embodiment, the input of ligand for the control device is also alternated as appropriate for the positive and negative selection cycles. In some embodiments, a drift cycle is used in between the positive and negative cycles. In some embodiments, a drift host cell containing a drift construct is placed in the chemostat while the level of ligand for the control device is changed to the desired level for the positive and negative selection cycle. In this embodiment, the drift construct encodes a polypeptide needed for propagation of the selection construct under control of a control region that is activated upon entry of the selection construct into the host cell. In some embodiments, the control region in the drift construct is a Pₚₛₚ promoter that is activated after infection of the host cell by the viral particle carrying the selection construct. In some embodiments, the drift host cell also contains a mutagenesis construct and during the drift cycle a desired number of selection construct mutants are made prior to the next selection cycle. In some embodiments, after the ligand has been washed out of the chemostat, the ligand has reached the desired level in the chemostat, and/or a desired number of selection construct mutants are obtained, the drift host cell is replaced by the corresponding positive or negative selection host cell.

In some embodiments, longer positive selection cycles are alternated with short negative selection cycles (using drift cycles in between). In this embodiment, desired positive selection mutants are obtained during the positive selection cycle, and certain unwanted mutants that also arise during the positive selection cycle are removed during the negative selection cycle (e.g., mutants that lose control device activity will be lost in the negative selection cycle). In some embodiments, longer negative selection cycles are alternated with short positive selection cycles. In some embodiments, the positive and negative selection hosts are placed in the chemostat at the same time. In some embodiments, the positive selection hosts further comprise a construct encoding an enzyme that degrades the ligand for the control device. In some embodiments, the negative selection host further comprises a construct encoding a transport protein that concentrates ligand for the control device into the negative selection host cell.

In some embodiments, the flow rates into and out of the chemostat are adjusted to desired levels to change the selective pressure on the selection construct to propagate. In some embodiments, the flow rates into and out of the chemostat are increased which increases the selection pressure for speed of propagation by the selection vector. In this embodiment, slower propagating selection constructs are washed out of the chemostat and only the faster propagating selection constructs remain. In some embodiments, the flow rates into and out of the chemostat are reduced which decreases the selection pressure for rapid propagation. In some embodiments, the flow rates are alternatively increased and decreased. In some embodiments, the slow flow rate conditions permissively allow slightly improved mutants to be made and then these mutants can be placed under high flow rate to select for mutants of these slightly improved mutants that have greater increased propagation rates. In some embodiments, this alternation of the flow rates allows multiple site mutants to arise that may not otherwise have been found under a constant high flow rate of selection.

In some embodiments, the ligand specificity of the control device is changed using positive and/or negative selection cycles of the invention. In some embodiments, the ligand for a control device is changed from theophylline to guanine using the positive selection of the invention. In this embodiment, the ligand added to the positive selection is guanine and positive selection will select for mutants of the theophylline binding control device that can bind guanine. In some embodiments, the ligand of a theophylline binding control device is first changed to xanthine by carrying out the positive selection in the presence of xanthine as the ligand. In some embodiments, the xanthine binding control device is then changed to guanine binding by adding guanine to the positive selection in place of xanthine. In some embodiments, the ligand specificity of a theophylline binding control device is changed to another purine, such as, for example, adenine, hypoxanthine, theobromine, caffeine, isoguanine, or uric acid. In some embodiments, the ligand specificity of a theophylline binding control device is changed to an antiviral drug, such as, for example, acyclovir.

In some embodiments, the host cells for the negative selection cycle (or the+ligand cycles) are engineered to include a SLC for increasing the concentration of ligand in the negative selection host cell. In some embodiments, the negative selection host cell is E coli, and the SLC is a purine permease selected from XanQ, XanP, PurP, UacT, YgfQ, YgfU, YicO, YbbY, YcjD, YgfO, or combinations of the foregoing SLCs. In some embodiments, the negative selection host cell is E coli, the ligand is xanthine, and the SLC engineered into the negative selection host cell is XanQ, XanP, YjcD, YgfO, YgfQ, YbbY, or combinations of the foregoing. In some embodiments, the negative selection host cell is E coli, the ligand is guanine, and the SLC engineered into the negative selection host cell is YgfQ, YjcD, YbbY, or combinations of the foregoing.

In some embodiments, the host cells for the positive selection cycle (or the minus ligand cycles) are engineered to include catabolic enzymes for the ligand or ligand modification enzymes which change the molecular structure of the ligand in the cell. In some embodiments, the change in structure to the ligand disrupts the binding of the ligand to the control device. In some embodiments, the negative selection host cell is E coli, and the purine modifying enzyme is xanthine dehydrogenase (xdhA, xdhB, xdhC), guanine deaminase (ygfP, guaD), and/or urate oxidase (A. flavus or B. subtilis). In some embodiments, the positive selection host cell is E coli, the ligand is xanthine, and the purine catabolic enzyme engineered into the positive selection host cell is xanthine dehydrogenase (xdhA, xdhB, xdhC) and/or urate oxidase (Uox of A. flavus or pucLM of B. subtilis). In some embodiments, the positive selection host cell is E coli, the ligand is guanine, and the purine catabolic enzyme engineered into the positive selection host cell is guanine deaminase (ygfP, guaD), xanthine dehydrogenase (xdhA, xdhB, xdhC), and/or urate oxidase (Uox of A. flavus or pucLM of B. subtilis).

In some embodiments, the negative selection host cell is E coli, and the SLC is a tetracycline (or tet derivative) transporter such as, for example, hsrA or yieO, or native transport proteins found in E. coli . In some embodiments, the negative selection host cell is E coli, the ligand is doxycycline, and the SLC engineered into the negative selection host cell hsrA or yieO, or native transport proteins found in E. coli . In some embodiments, the negative selection host above is used to select for control devices that have changed ligand specificity from tetracycline to doxycycline, or have become sensitive to doxycycline (and still have sensitivity to tetracycline). In some embodiments, the negative selection is used to increase the sensitivity of the control device to doxycycline (or tetracycline) by demanding that the control device be able to turn off activity at lower concentrations of ligand (doxycycline and/or tetracycline).

In some embodiments, positive and negative selection host cells are used at the same time by engineering the positive selection host cells with ligand degradation enzymes, and the negative selection host cells with permeases for the ligand. In this embodiment, the positive selection host cells will have a lower concentration of ligand as compared to the negative selection host cells. In some embodiments, an optimal range of ligand is determined for growth and simultaneous selection in the positive selection host cells (ligand degradation) and negative selection host cells (permease). In some embodiments, the ligand for the control device is xanthine, the negative selection host is engineered to express XanQ, XanP, YjcD, YgfO, YgfQ, YbbY, or combinations of the foregoing, and the positive selection host is engineered to express xanthine dehydrogenase (xdhA, xdhB, xdhC) and/or urate oxidase (A. flavus or B. subtilis). In this embodiment, the xanthine dehydrogenase expressed in the positive selection host degrades xanthine to uric acid and the urate oxidase degrades the uric acid to allantoin (a metabolic intermediate in E. coli) and the control device is active. Also in this embodiment, and the XanQ, XanP, YjcD, YgfO, YgfQ, and/or YbbY permeases import xanthine into the negative selection host cells increasing the concentration of xanthine in the cell so that the control device is inactive (or inhibited). In some embodiments, the selection construct moves between the positive and negative selection hosts and undergoes simultaneous negative and positive selection.

In some embodiments, multiple positive selection and/or negative selection cycles are performed in parallel. In some embodiments, multiple positive selection cycles are performed in parallel. In some embodiments multiple negative selection cycles are performed in parallel. In some embodiments, multiple positive and negative selection cycles are performed in parallel. In some embodiments, multiple positive-negative cycles (simultaneous positive and negative selection hosts) are performed in parallel. In some embodiments, the selection constructs from the multiple selection cycles are mixed together and subjected to a positive and/or negative selection cycle(s). In this embodiment, the best or most fit selection construct will be selected from the pooled cycle(s). In some embodiments, selection constructs from one selection cycle is pooled with the selection construct of one other selection cycle. In this embodiment, the best or most fit selection construct from the two cycles (or pools) will be selected. In some embodiments, this winning selection construct is then pooled with another winning construct from the mixture of other selection constructs from two other cycles (pools). In some embodiments, this competition between selection constructs is continued until a desired number of winning constructs is obtained.

In some embodiments, the different cycles (pools) are combined in a single elimination tournament style, where a winning selection construct arises from each pair of cycles (pools) and the losing construct is eliminated from consideration. In this embodiment, a winning construct is then paired with another winning construct and so one until only one winning construct is obtained. In some embodiments, aliquots from one cycle (pool) are mixed pairwise with aliquots from other pools in a round robin fashion. In this embodiment, the winning selection constructs form the round robin can be paired together in further round robin cycles, or can be placed into a tournament style competition to arrive at one winning construct, or round robin pools can be combined to produce a single (or a few) winning selection constructs.

Process for Producing Eukaryotic Cells Expressing a Transgene Under the Control of a Ligand Matched Control Device, Control Region, and/or SLC

A process for producing a cell expressing a transgene under the control of ligand matched control devices, control regions, and SLCs includes a step of introducing the nucleic acid encoding the Control Region-Control Device-Transgene-Construct and the nucleic acid encoding the SLC into a eukaryotic cell. In some embodiments, this step is carried out ex vivo. For example, a cell can be transformed ex vivo with a virus vector or a non-virus vector carrying the nucleic acid of the present invention to produce a cell expressing the control region—control device—transgene and optionally a ligand matched SLC of the present invention. In some embodiments, the nucleic acid also includes a ligand matched, inducible control region, wherein the control region expressing the transgene is regulated by the same ligand (either induction or repression of the control region). Examples of inducible control regions that can be used in the invention can be found in Kis et al., J. R. Soc. Interface 12:20141000 (2015), and Auslander et al, Treand in Biotechnol. 31:155-168 (2012), both of which are incorporated by reference in their entirety for all purposes.

In the process of the present invention, a eukaryotic cell as described above is used. In some embodiments, a eukaryotic cell derived from a mammal, for example, a human cell, or a cell derived from a non-human mammal such as a monkey, a mouse, a rat, a pig, a horse, or a dog can be used. The cell used in the process of the present invention is not particularly limited, and any cell can be used. For example, a cell collected, isolated, purified or induced from a body fluid, a tissue or an organ such as blood (peripheral blood, umbilical cord blood etc.) or bone marrow can be used. A peripheral blood mononuclear cell (PBMC), an immune cell, a dendritic cell, a B cell, a hematopoietic stem cell, a macrophage, a monocyte, a NK cell or a hematopoietic cell, an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In the present invention, particularly, use of a T cell, a precursor cell of a T cell (a hematopoietic stem cell, a lymphocyte precursor cell etc.) or a cell population containing them is preferable. Examples of the T cell include a CD8-positive T cell, a CD4-positive T cell, a regulatory T cell, a cytotoxic T cell, and a tumor infiltrating lymphocyte. The cell population containing a T cell and a precursor cell of a T cell includes a PBMC. The aforementioned cells may be collected from a living body, obtained by expansion culture of a cell collected from a living body, or established as a cell strain. When transplantation of the produced Smart-Construct or DE-Construct expressing cell or a cell differentiated from the produced Smart-Construct or DE-Construct expressing cell into a living body is desired, it is preferable to introduce the nucleic acid into a cell collected from the living body itself.

In an embodiment, the nucleic acids encoding the control region-control device-transgene and SLC are integrated into the eukaryotic cell chromosome at a genomic safe harbor site, such as, for example, the CCR5, AAVS1, human ROSA26, or PSIP1 loci. (Sadelain et al., Nature Rev. 12:51-58 (2012); Fadel et al., J. Virol. 88(17):9704-9717 (2014); Ye et al., PNAS 111(26):9591-9596 (2014), all of which are incorporated by reference in their entirety for all purposes.) In an embodiment, the integration of the nucleic acid encoding the control device-transgene and SLC at the CCR5 or PSIP1 locus is done using a gene editing system, such as, for example, CRISPR, TALEN, or Zinc-Finger nuclease systems. In an embodiment, the eukaryotic cell is a human, T-lymphocyte and a CRISPR system is used to integrate the Smart-Construct or DE-Construct at the CCR5 or PSIP1 locus. In an embodiment, integration of the nucleic acid at CCR5 or PSIP1 using the CRISPR system also deletes a portion, or all, of the CCR5 gene or PSIP1 gene. In an embodiment, Cas9 in the eukaryotic cell may be derived from a plasmid encoding Cas9, an exogenous mRNA encoding Cas9, or recombinant Cas9 polypeptide alone or in a ribonucleoprotein complex. (Kim et al (2014) Genome 1012-19. doi:10.1101/gr.171322.113.; Wang et al (2013) Cell 153 (4) Elsevier Inc.: 910-18. doi:10.1016/j.ce11.2013.04.025, both of which are incorporated by reference in their entirety for all purposes.)

Methods for introducing the nucleic acids of the invention into appropriate host cells are well known in the art, including those described in U.S. patent application Ser. No. 15/070,352 filed on Mar. 15, 2016, which is incorporated by reference in its entirety for all purposes.

In some embodiments, chemical structures with the ability to promote stability and/or translation efficiency are used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA. The 5′ and 3′ UTRs can be the naturally occurring, endogenous 5′ and 3′ UTRs for the nucleic acid of interest. In some embodiments, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

### Eukaryotic Cells Expressing Transgenes Under the Control of Ligand Matched Control Regions, Control Devices and SLCs

The cell expressing the control region-control device-transgene and SLC of the present invention is a cell in which nucleic acids encoding control region-control device-transgene and SLC are introduced and expressed. In some embodiments, the construct includes a ligand matched control region so that the same ligand controls expression of the transgene from the control region, from the control device, and is imported by the SLC. In some embodiments, the same ligand controls expression of the transgene from the control region and from the control device. In some embodiments, a control device regulates the expression of the protein that acts at the control region. In some embodiments, this control device is also ligand matched with the other control elements.

In some embodiments, the ligand matched control devices, transcription regulators (e.g., activator or repressor) and/or solute carriers are used to control the expression of a transgene that is engineered into a eukaryotic cell. In some embodiments, the ligand matched control devices, transcription regulators (e.g., activator or repressor) and/or solute carriers are used to control the expression of a transgene encoding a CAR. In some embodiments, the ligand matched control devices, transcription regulators (e.g., activator or repressor) and/or solute carriers are used to control the expression of a transgene encoding a cytokine. In some embodiments, the ligand matched control devices, transcription regulators (e.g., activator or repressor) and/or solute carriers are used to control the expression of a transgene encoding an effector polypeptide. In some embodiments, the ligand matched control devices, transcription regulators (e.g., activator or repressor) and/or solute carriers are used to control the expression of a transgene encoding a polypeptide for gene therapy. In some embodiments, the ligand matched control devices, transcription regulators (e.g., activator or repressor) and/or solute carriers are used to control the expression of a transgene encoding a transcriptional factor. In some embodiments, the ligand matched control devices, transcription regulators (e.g., activator or repressor) and/or solute carriers are used to control the expression of a transgene encoding a signal transduction polypeptide. In some embodiments, the ligand matched control devices, transcription regulators (e.g., activator or repressor) and/or solute carriers are used to control the expression of a transgene encoding a receptor. In some embodiments, the ligand matched control devices, transcription regulators (e.g., activator or repressor) and/or solute carriers are used to control the expression of a transgene encoding a secreted polypeptide. In some embodiments, the ligand matched control devices, transcription regulators (e.g., activator or repressor) and/or solute carriers are used to control the expression of a transgene encoding an enzyme. In some embodiments, the ligand matched control devices, transcription regulators (e.g., activator or repressor) and/or solute carriers are used to control the expression of a transgene encoding a structural polypeptide.

In some embodiments, the ligand matched control devices, transcription regulators (e.g., activator or repressor) and/or solute carriers are used to control the expression of a transgene useful in therapies to treat, for example, amyotrophic lateral sclerosis (ALS), type I diabetes, Parkinson's disease, Alzheimer's, cardiac diseases, osteoarthritis, cancer, stroke, and wound repair. In some embodiments, the ligand matched control devices, transcription regulators (e.g., activator or repressor) and/or solute carriers are used to control the expression of a transgene useful in gene replacement therapies to treat, for example, hemophilia, β-thalassemia, Sanfilippo syndrome, macular degeneration, cystic fibrosis, amyotrophic lateral sclerosis (ALS), severe combined immunodeficiency (SCID), and chronic granulomatous disease.

In some embodiments, the ligand matched control devices, transcription regulators (e.g., activator or repressor) and/or solute carriers are used to control the expression of a CAR, DE-CAR, or Side-CAR useful in treating a malignancy including, for example, a solid tumor, a leukemia, lymphoma, myeloma, myelodysplastic syndrome, and/or myeloproliferative disease. In some embodiments, the malignancy is a multiple myeloma. In some embodiments, the malignancy is, for example, a sarcoma, carcinoma, melanoma, or blastoma. In some embodiments, the malignancy is a cancer of, for example, the adrenal glands, bile ducts, bladder, bone, brain-CNS, breast, cervix, colorectum, endometrial, esophagus, eye, gallbladder, gastrointestinal, kidney, larynx, liver, lung, nasal cavity, ovary, pancreas, pituitary prostate, salivary gland, skin, stomach, testicular, thymus, and uterine. In some embodiments, the malignancy is a CD19 and/or CD20 positive B-cell lymphoma. In some embodiments, the ligand matched control devices, transcription regulators (e.g., activator or repressor) and/or solute carriers are used to control the expression of a CAR, DE-CAR, or Side-CAR useful in treating an autoimmune disease, such as, for example a neurological disorder (e.g., multiple sclerosis), a rheumatological disorder (e.g., rheumatoid arthritis, systemic sclerosis, systemic lupus), a hematological immunocytopenia (pure red cell aplasia, immune thrombopenia, pure white cell aplasia), or a gastrointestinal disorder (inflammatory bowel disease). Other diseases that may be treated with eukaryotic cells expressing the Smart CAR, DE-CAR, Smart-DE-CAR, and/or Side CAR are disclosed in U.S. patent application Ser. No. 15/070,352 filed on Mar. 15, 2016, which is incorporated by reference in its entirety for all purposes.

In some embodiments, a eukaryotic cell expressing the control device-transgene, transcription regulator, and/or SLC are used as a therapeutic agent to treat a disease. In some embodiments, the control region-transcription activator is the Tet-On control region and the Tet activator, the control device is a Tet sensitive ribozyme, e.g., Beilstein et al., ACS Synth. Biol. 4:526-534 (2016), which is incorporated by reference in its entirety for all purposes, and the SLC is a tetracycline transporter (e.g., hOAT1, hOAT2, hOAT3, hOAT4, SLC22A6, SLC22A7, SLC22A11). The therapeutic agent comprises the eukaryotic cell expressing the control region-control device-transgene and/or SLC as an active ingredient, and may further comprise a suitable excipient. Examples of the excipient include pharmaceutically acceptable excipients for the composition. The disease against which the eukaryotic cell expressing the control device-transgene and SLC are administered is not particularly limited as long as the disease shows sensitivity to the eukaryotic cell.

The eukaryotic cell expressing the control region-control device-transgene and/or SLC of the present invention is administered for treatment of these diseases. The eukaryotic cell of the present invention can also be utilized for prevention of an infectious disease after bone marrow transplantation or exposure to radiation, donor lymphocyte transfusion for the purpose of remission of recurrent leukemia, and the like. The therapeutic agent comprising the eukaryotic cell expressing the Smart CAR, DE-CAR, Smart-DE-CAR, and/or Side CAR as an active ingredient can be administered intradermally, intramuscularly, subcutaneously, intraperitoneally, intranasally, intraarterially, intravenously, intratumorally, or into an afferent lymph vessel, by parenteral administration, for example, by injection or infusion, although the administration route is not limited.

In some embodiments, the eukaryotic cells with control region, control device-transgene and/or SLC are characterized prior to administration to the subject. In some embodiments, the eukaryotic cells with control device-transgene and SLC are tested to confirm control device-transgene and SLC expression. In some embodiments, the eukaryotic cells with control region, control device-transgene and SLC are exposed to a level of ligand(s) that results in a desired level of transgene expression in the eukaryotic cell. In some embodiments, this desired level of transgene produces eukaryotic cells with a desired level of transgene activity when placed in a subject.

In some embodiments, a ligand matched SLC, control region, and/or Smart CAR, DE-CAR, Smart-DE-CAR, and/or Side CAR is used with a T-lymphocyte that has aggressive anti-tumor properties, such as those described in Pegram et al, CD28z CARs and armored CARs, 2014, Cancer J. 20(2):127-133, which is incorporated by reference in its entirety for all purposes.

In some embodiments, the control region, control device-transgene and/or SLC of the invention are used to control the expression of gene therapy constructs. In some embodiments, the novel control devices of the invention are used to control the expression of recombinant gene products expressed in a cell.

In some embodiments, nucleic acids encoding control regions, control device-transgenes and/or SLCs of the invention are used to express control device-transgene and SLC polypeptides in mammalian cells. In some embodiments, nucleic acids encoding control region, control device-transgene and/or SLC of the invention are used to express control device-transgene and SLC polypeptides in human cells or murine cells. In some embodiments, nucleic acids encoding control region, control device-transgenes and/or SLCs of the invention are used to express control device-transgene and SLC polypeptides in hematopoietic cells. In some embodiments, nucleic acids encoding the control regions, control device-transgenes and/or SLCs of the invention are used to express control device-transgene and SLC polypeptides in T-cells, natural killer cells, B-cells, or macrophages. In some embodiments, nucleic acids encoding control region, control device-transgenes and/or SLCs of the invention are used to express control device-transgene and SLC polypeptides in T-cells or natural killer cells.

In some embodiments, the ligand matched novel control regions, control devices and/or SLCs of the invention are used to make nucleic acids encoding a construct to be expressed in a cell for gene therapy. In some embodiments, these control region, control device-transgene and/or SLC constructs are used for gene therapy in mammalian cells. In some embodiments, these control region, control device-transgene and/or SLC constructs are used for gene therapy in human cells or murine cells. In some embodiments, these control region, control device-transgene and/or SLC constructs are used for gene therapy in human cells.

In some embodiments, the ligand matched control regions, control devices and/or SLCs of the invention are used to make nucleic acids encoding a construct to be expressed in a cell. In some embodiments, these control region, control device-transgene and/or SLC constructs are used to express polypeptides in mammalian cells. In some embodiments, these control region, control device-transgene and/or SLC constructs are used to express polypeptides in human cells or murine cells. In some embodiments, ligand matched control regions, control devices, and/or SLCs are used to make nucleic acids encoding a construct to be expressed in a cell. In some embodiments, ligand matched control regions, control device-transgenes, and/or SLC constructs are used to express polypeptides in mammalian cells. In some embodiments, these control region, control device-transgene and/or SLC constructs are used to express polypeptides in human cells or murine cells.

In an exemplary embodiment, a TET-ON inducible promoter, a Tet controlled RNA control device (or DE or RDE), and a Tet SLC (e.g., hOAT1, hOAT2, hOAT3, hOAT4, SLC22A6, SLC22A7, or SLC22A11) are used to control expression of a transgene. In some embodiments, the TET-ON inducible promoter is engineered (e.g., using Cheetah) to reduce the constitutive level of expression from the control region, and/or increase the off-rate of tetracycline binding by the Tet-activator, and/or reduce the on-rate of the Tet-activator. In some embodiments, the Tet-activator is engineered to reduce its immunogenicity in mammals. For example, the Tet-activator could be engineered with glycosylation sites to provide the polypeptide with a hydration shield that should reduce immuno-detection and immunoreactivity. In this all Tet induced control system, the dynamic range of expression in response to the ligand tetracycline should be increased.

In some embodiments, the Tet induced control system (or other analogous ligand matched control system) is used to express a transgene of interest in a eukaryotic cell. In this embodiment, tetracycline binds to the Tet-activator to induce transcription, binding of tetracycline by the RNA control device reduces ribozyme activity, and/or binding of tetracycline by the DE increases stability of the polypeptide containing the DE. In some embodiments, the Tet sensitive RNA control device or DE is used to control the expression of the Tet-activator. In some embodiments, multiple (two or more) copies of the Tet (or other ligand) RNA control device are placed into the construct to increase the dynamic range of control by the RNA control device. Increasing the number of copies of the RNA control device reduces the basal level of expression in the system without ligand.

In some embodiments, the nucleic acids encoding the control device-transgene and SLC constructs of the invention are used to express a desired level of a desired polypeptide (e.g., for gene therapy, or a CAR, DE-CAR, and/or Side-CAR polypeptide). In some embodiments, the ligand matched control device and SLC control the level of desired polypeptide expression, at least in part, and by modulating the level of activity of the control device. In some embodiments, the control device increases the degradation rate of DE-polypeptide in the eukaryotic cell and when ligand is bound by the DE, the rate of degradation decreases. In some embodiments, the DE increases degradation of the DE-polypeptide when ligand is bound by the DE. In some embodiments, the RNA control device inhibits translation of the DE-polypeptide mRNA and when ligand binds to the sensor element of the RNA control device this inhibition of translation is reduced so that polypeptide expression is increased. In some embodiments, ligand for the DE and/or the ligand for the RNA control device sensor is added in increasing amounts to the eukaryotic cells with the Smart-construct and/or DE-construct until a desired level of polypeptide is made in the eukaryotic cell.

In some embodiments, the ligand for the DE and/or the ligand for the RNA control device sensor is added in increasing amounts until a desired level of activity is obtained. In some embodiments, the desired eukaryotic cell activity occurs over a desired time period, e.g., a certain amount of activity in 12 hours, or 24 hours, or 36 hours, or two days, or 3, 4, 5, 6, 7, 8, 9, 10, 11, 12, 13, 14, 15, 16, 17, 18, 19, 20, 21, 22, 23, 24, 25, 26, 27, or 28 days, or two months, or 3, 4, 5, or 6 months. In some embodiments, activity is expressed as a half-life. In this embodiment, the half-life can be 12 hours, 24 hours, 36 hours, or 2, 3, 4, 5, 6, 7, 8, 9, 10, 11, 12, 13, 14, 15, 16, 17, 18, 19, 20, 21, 22, 23, 24, 25, 26, 27, or 28 days, or two months, or 3, 4, 5, or 6 months.

In some embodiments, a regime of different amounts of ligand (for the control device) is added over time so that different desired levels of polypeptide are present in the eukaryotic cell at different times. For example, during the enrichment/selection of antigen binding domains with Smart CAR, DE-CAR, Smart-DE-CAR, and/or Side CAR T-cells or Smart CAR, DE-CAR, Smart-DE-CAR, and/or Side CAR natural killer cells, the amount of CAR, DE-CAR, and/or Side-CAR polypeptide expression may be relatively high initially to ensure that some antigen binding domains are able to bind target antigen, and in subsequent rounds of enrichment/selection, the amount of CAR, DE-CAR, and/or Side-CAR polypeptide expression can be decreased to enrich/select for antigen binding domains with higher affinity for target antigen. In some embodiments, gene therapy expression may be relatively low initially until the engineered cell reaches a target location or a set of conditions is satisfied at which time polypeptide expression is increased.

In some embodiments the nucleic acid sequences encoding a control device are present in a nucleic acid locus encoding a transgene. In some embodiments, RNA control devices are encoded for as nucleic acid sequence in the vector proximal, distal, or within the ORF encoding a transgene polypeptide. In some embodiments nucleic acid sequences encoding an RNA control device or devices are located within the 3′ UTR region of the transgene. In some embodiments nucleic acid sequences encoding an RNA control device or devices are located in the 5′ UTR region of the transgene. In some embodiments nucleic acid sequences encoding an RNA control device or devices are located within synthetic or natural introns flanked by coding or noncoding exons within the transgene, or at intron/exon boundaries.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1

### Positive Selection for a Control Device

A selection construct is made using the RNA control device, 3XL2bulge9 (Win and Smolke 2007 Proc. Natl Acad. Sci. 104 (36): 14283-88, which is hereby incorporated by reference in its entirety for all purposes) which is engineered into a construct so that expression of T7 RNA polymerase is under the control of the RNA control device. The selection construct also includes a constitutive promoter for expressing mRNA encoding the RNA control device and T7 RNA polymerase, an f1 origin of replication and packaging signal region, the bla gene, and an ori for replication in host cells during the engineering steps. The selection construct makes a mRNA transcript for T7 RNA polymerase that is cleaved by the RNA control device in the absence of ligand (theophylline). When theophylline is added, this ligand binds to the RNA control device and inhibits the ribozyme activity of the RNA control device allowing the T7 RNA polymerase to be translated from the mRNA.

A M13 helper construct is used that has a defective pIII gene so that the helper construct does not make pIII that is functional for infecting host cells. Such pIII defective helper constructs are well known in the art including, for example, the M13KO7d3 helper phage that is commercially available from Antibody Design Laboratories, or the VCSM13 helper phage described in Kramer et al., Nucl. Acids Res. 31:e59 (2003), which are incorporated by reference in its entirety for all purposes.

A positive selection construct is made in which a polynucleotide encoding the M13 pIII protein is placed under the control of a T7 promoter. This construct will express functional pIII protein when T7 RNA polymerase is present in the host cell.

A mutagenesis construct is used, such as those described in Badran et al, Nat. Commun. 6:8425 (2015), which is incorporated by reference in its entirety for all purposes. For example, the MP6 construct expressing dnaQ926, dam, seqA, emrR, ugi, and cdal from Badran 2015 is used.

A SLC construct is made that constitutively expresses the XanQ, XanP, YjcD, YgfO, YgfQ, YicE, and/or YbbY genes which encode SLCs that transport xanthine. A second SLC construct is made that constitutively expresses the ghxQ and/or ghxP genes which encode SLCs that transport hypoxanthine.

An E. coli host cell containing the helper construct and positive selection construct is made. Alternative E. coli host cells are made with the helper construct, positive selection construct, and optionally the mutagenesis construct, and optionally the SLC construct.

The selection construct is placed into an E. coli host cell (helper and positive selection constructs). Xanthine is added to the cells, and RNA control devices that can bind xanthine and inhibit the ribozyme function will package selection construct into phage particles that can infect other E. coli host cells. Selection constructs with RNA control devices that cannot bind xanthine will not make phage particles and will be lost from the selection. In some embodiments, xanthine is initially added at a high concentration, and at later selection steps, the xanthine concentration is decreased to select or enrich for those RNA control devices that produce a more robust off signal in response to xanthine.

The selection construct is also placed into an alternate E. coli host cell (helper, positive selection, and mutagenesis constructs). Xanthine is added to the cells, and RNA control devices that can bind xanthine and inhibit the ribozyme function will package selection construct into phage particles that can infect other alternate E. coli host cells. Selection constructs with RNA control devices that cannot bind xanthine will not make phage particles and will be lost from the selection. In some embodiments, xanthine is initially added at a high concentration, and at later selection steps, the xanthine concentration is decreased to select or enrich for those RNA control devices that produce a more robust off signal in response to xanthine.

The selection construct is placed into a second alternate E. coli host cell (helper, positive selection, mutagenesis, and permease constructs). Xanthine is added to the cells, and RNA control devices that can bind xanthine and inhibit the ribozyme function will package selection construct into phage particles that can infect other second alternate host cells. Selection constructs with RNA control devices that cannot bind xanthine will not make phage particles and will be lost from the selection. In some embodiments, xanthine is initially added at a high concentration, and at later selection steps, the xanthine concentration is decreased to select or enrich for those RNA control devices that produce a more robust off signal in response to xanthine.

### Example 2

### Negative Selection for a Control Device

The selection construct, helper construct, and mutagenesis construct from Example 1 are used in this example.

A drift construct is made with a polynucleotide encoding pIII under the control of a phage shock promoter (Pₚₛₚ) which is activated to express pIII upon infection of the cell by a phage particle with the selection construct.

A negative selection construct is made with a polynucleotide encoding a N-C83 mutant of pIII (Bennett et al., J. Mol. Biol. 356:266-73 (2005), which is incorporated by reference in its entirety for all purposes) under the control of a T7 promoter. The N-C83 pIII mutant makes phage particles that are unable to infect new host cells. The N-C83 pIII protein is made when there is T7 RNA polymerase in the host cell.

A purine catabolic construct is made that constitutively expresses xanthine dehydrogenase (xdhA, xdhB, xdhC) and optionally expresses urate oxidase (Uox of A. flavus or pucLM of B. subtilis).

A negative selection host cell is made using E. coli containing the helper construct, the drift construct, and the negative selection construct. An alternative negative selection host is made using E. coli containing the helper construct, the drift construct, the negative selection construct, and the mutagenesis construct. A second alternative negative selection host is made using E. coli containing the helper construct, the drift construct, the negative selection construct, the mutagenesis construct, and the purine catabolic construct.

The selection construct is packaged into a phage particle and these are used to infect a negative selection host cell (helper construct, drift construct, and negative selection construct). RNA control devices that inhibit expression of T7 RNA polymerase will package selection construct into infective phage particles that can infect other negative selection host cells. Selection constructs with RNA control devices that do not inhibit mRNA translation (by cleavage) are lost as T7 RNA polymerase is made in these host cells and noninfective phage particles are made (phage particles made with N-C83 pIII). In some embodiments, theophylline is added to the cells, and this selects or enriches for RNA control devices that no longer respond to theophylline as the ligand.

Phage particles with the selection construct are also used to infect alternative negative selection host cells. In this embodiment, RNA control devices that inhibit expression of T7 RNA polymerase will package selection construct into infective phage particles that can infect other negative selection host cells. Selection constructs with RNA control devices that do not inhibit mRNA translation (by cleavage) are lost as T7 RNA polymerase is made in these host cells and noninfective phage particles are made (phage particles made with N-C83 pIII). In some embodiments, theophylline is added to the cells, and this selects or enriches for RNA control devices that no longer respond to theophylline as the ligand.

Phage particles with the selection construction are used to infect second alternative negative selection host cells. In this embodiment, RNA control devices that inhibit expression of T7 RNA polymerase will package selection construct into infective phage particles that can infect other negative selection host cells. Selection constructs with RNA control devices that do not inhibit mRNA translation (by cleavage) are lost as T7 RNA polymerase is made in these host cells and enoninfective phage particles are made (phage particles made with N-C83 pIII). In some embodiments, theophylline is added to the cells, and this selects or enriches for RNA control devices that no longer respond to theophylline as the ligand.

### Example 3

### Drift Host Cells

This example uses the selection construct and helper construct from Example 1 and the drift construct from Example 2. Optionally, the drift host cells also contain the mutagenesis construct.

Drift host cells are made using E. coli containing the helper construct from Example 1 and the drift construct from Example 2. When selection constructs infect drift host cells the drift construct makes pIII polypeptide and in combination with the helper construct new M13 particles are made for packaging the selection construct. Drift host cells allow for control device ligand to be added or washed out between positive and negative selection cycles.

Optionally, prior to the positive or negative selections of Examples 1 or 2, selection constructs are grown in drift host cells that also contain the mutagenesis construct to generate a diverse population of control device mutants prior to the positive and/or negative selection cycle.

### Example 4

### Positive and Negative Selection Cycles Performed in Series

The selection construct of Example 1 is cycled through a positive selection in a chemostat as described in Example 1. During the positive selection cycle, media with ligand (xanthine) and positive selection host cells (Example 1) are added to the chemostat. At the end of the cycle, media (without ligand) with drift host cells (Example 3 which optionally contain the mutagenesis construct) is added to the chemostat. After ligand is washed out of the chemostat (or after generation of desired number of mutants), media with negative selection host cells (Example 2) is added to the chemostat. Optionally, theophylline is added to the media with the negative selection host cells to select for control devices that have changed specificity from theophylline to xanthine.

After 30-50 positive and negative cycles in the growth vessel, selection constructs with the control devices that bind xanthine are isolated.

### Example 5

### Simultaneous Positive and Negative Selection Cycles

The selection construct of Example 1 is packaged into M13 viral capsids which are added to media having ligand (xanthine), second alternative positive selection host cells from Example 1 (host cells containing helper constructs, positive selection constructs, permease constructs, and optionally mutagenesis constructs), and second alternative negative selection host cells from Example 2 (host cells containing helper constructs, drift constructs, negative selection constructs, purine catabolic constructs, and optionally mutagenesis constructs).

Optionally, the simultaneous selection is performed in a chemoset, and after 30-50 generations of growth, media is added to the growth vessel having ligand (xanthine) and just positive selection host cells. Optionally, the xanthine concentration may be lowered to place higher selection stringency upon the population.

Also optionally, the selection construct is cycled through drift cycles with a drift host cell (Example 3) that contains the mutagenesis construct to generate a population of mutant control devices prior to the next round of simultaneous selection.

Selection constructs are harvested from the chemostat and control devices with xanthine sensitivity are isolated.

### Example 6

### Ligand Matched RNA Control Device and SLC

The xanthine sensitive RNA control device obtained in Examples 1-5 above, is paired with an SLC transporter that has substrate specificity for xanthine (see above). Nucleic acids encoding this RNA control device are operably linked to nucleic acids encoding a CD20 CAR (Budde et al. PLoS1, 2013 doi:10.1371/journal.pone.0082742, which is hereby incorporated-by-reference in its entirety for all purposes). Nucleic acids encoding the xanthine SLC transporter, and nucleic acids encoding the RNA control device—anti-CD20 CAR are engineered into T-lymphocytes.

This anti-CD20 CAR and SLC T-lymphocytes are activated by co-incubation with anti-CD3/CD28 beads. Activated anti-CD20 CAR and SLC T-lymphocytes T-cells are co-cultured with CD20+target cells at anti-CD20 CAR and SLC T-lymphocytes:CD20+target ratios of 2:1, 5:1, and 10:1. Ligand for the RNA control device, xanthine is added to the culture medium at concentrations in the range of 5 μM to 1 mM (lower or greater concentrations can be used to titrate Smart-CAR activity to the desired level). The anti-CD20 CAR and SLC T-lymphocytes and target cells are grown together for 48 hours. Cultures are washed, and then stained with anti-CD20 and anti-CD3 reagents, followed by counting of CD20+ (target cells) and CD3+ cells (anti-CD20 CAR and SLC T-lymphocytes). These measurements will identify the target cell killing rate (e.g., half-life) and the proliferation rate of the anti-CD20 CAR and SLC T-lymphocytes at different levels of CAR.

### Example 7

### Ligand Matched Control Region, RNA Control Device and SLC

A tetracycline sensitive control device (Beilstein et al. ACS Synth. Biol. 4:526-534 (2016), which is incorporated by reference in its entirety for all purposes) is combined with the TET-ON control region and transcription activator (systems commercially sold by Takara Bio Co.) to control expression of an anti-CD20 CAR described in the previous examples. An expression construct is made wherein the expression of the anti-CD20 CAR is under the control of the tetracycline activated control region (Tet activator) and the tetracycline repressible RNA control device. In an alternative embodiment, tandem RNA control devices are engineered into the construct to increase the inhibition of expression from the RNA control device. This DNA construct is placed in a T-lymphocyte along with a construct encoding a tetracycline transporter, such as, SLC22A6, SLC22A7, and/or SLC22A11.

The anti-CD20 CAR construct and SLC T-lymphocytes are activated by co-incubation with anti-CD3/CD28 beads. Activated anti-CD20 CAR and SLC T-lymphocytes T-cells are co-cultured with CD20+ target cells (e.g., Raji B-cells) at 1:1 CAR and SLC T-lymphocytes:CD20+ target ratios of 2:1, 5:1, and 10:1. Ligand for the control region, RNA control device, and SLC, tetracycline, is added to the culture medium at concentrations in the range of 5 μM to 1 mM (lower or greater concentrations can be used to titrate Smart-CAR activity to the desired level). The anti-CD20 CAR and SLC T-lymphocytes and target cells are grown together for 48 hours. Cultures are washed, and then stained with anti-CD20 and anti-CD3 reagents, followed by counting of CD20+ (Raji B cells) and CD3+ cells (anti-CD20 CAR and SLC T-lymphocytes). These measurements will identify the target cell killing rate (e.g., half-life) and the proliferation rate of the anti-CD20 CAR and SLC T-lymphocytes at different levels of CAR.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

What is claimed is:

1. A nucleic acid comprising: a polynucleotide encoding a RNA control device operably linked to a polynucleotide encoding a CAR, wherein the CAR is comprised of an extracellular element, a transmembrane element, and an intracellular element, and wherein the transmembrane element is between the extracellular element and the intracellular element, and a polynucleotide encoding a solute carrier transporter wherein the ligand of the control device is transported by the solute carrier transporter.

2. The nucleic acid of claim 1, wherein the RNA control device is comprised of a sensor element and a regulatory element.

3. The nucleic acid of claim 1, further comprising a control region, wherein the control region is operably linked to the polynucleotide encoding the CAR and the polynucleotide encoding the RNA control device, and wherein expression from the control region is induced by the ligand.

4. The nucleic acid of claim 3, wherein the polynucleotide encoding the CAR further comprises a polynucleotide encoding a costimulatory element, wherein the transmembrane element is between the extracellular element and the costimulatory element.

5. A nucleic acid comprising: a polynucleotide encoding a RNA control device operably linked to a polynucleotide encoding a transgene, and a polynucleotide encoding a solute carrier transporter wherein the ligand of the control device is transported by the solute carrier transporter.

6. The nucleic acid of claim 5, wherein the RNA control device is comprised of a sensor element and a regulatory element.

7. The nucleic acid of claim 5, further comprising a control region, wherein the control region is operably linked to the polynucleotide encoding the transgene and the polynucleotide encoding the RNA control device, and wherein expression from the control region is induced by the ligand.

8. A mammalian cell comprising the nucleic acid of claim 1.

9. A mammalian cell comprising the nucleic acid of claim 4.

10. A mammalian cell comprising the nucleic acid of claim 5.

11. A mammalian cell comprising the nucleic acid of claim 7.

12. The mammalian cell of claim 8, wherein the mammalian cell is a human cell.

13. The mammalian cell of claim 10, wherein the mammalian cell is a human cell.

14. The mammalian cell of claim 8, wherein the mammalian cell is a hematopoietic cell.

15. The mammalian cell of claim 10, wherein the mammalian cell is a hematopoietic cell.

16. The mammalian cell of claim 8, wherein the mammalian cell is a T-lymphocyte.

17. The mammalian cell of claim 9, wherein the mammalian cell is a T-lymphocyte.

18. The mammalian cell of claim 10, wherein the mammalian cell is a T-lymphocyte.

19. The mammalian cell of claim 8, wherein the mammalian cell is a natural killer cell.

20. The mammalian cell of claim 10, wherein the mammalian cell is a natural killer cell.
